(19) United States
(12) Patent Application Publication
(10) Pub. No.: US 2022/0002819 A1
(43) Pub. Date: Jan. 6, 2022

(54) INTRAGENIC ASSESSMENT AND METHODS THEREFOR

(71) Applicant: THE UNIVERSITY OF SYDNEY, Sydney - New South Wales (AU)

(72) Inventor: Sandra COOPER, Sydney (AU)

(21) Appl. No.: 17/436,044

(22) PCT Filed: Mar. 13, 2020
(86) PCT No.: PCT/AU2020/050234

§ 371 Date: Sep. 2, 2021

(30) Foreign Application Priority Data
Mar. 13, 2019 (AU) 2019900836

(51) Int. Cl. C12Q 1/6886

(52) U.S. Cl. CPC C12Q 1/6886; C12Q 2600/158; C12Q 2600/156

(57) ABSTRACT

A method for determining the likelihood that a genetic variant of a genetic locus defines a genetic disease or cancer-associated allele.

### FIELD OF THE INVENTION

The invention relates to identification of genetic markers of disease, and to use of markers for determining the likelihood of a disease or a condition.

### RELATED APPLICATION

This application claims priority from Australian provisional application AU 2019900836, the entire contents of which are hereby incorporated by reference.

### BACKGROUND OF THE INVENTION

Massively parallel sequencing is transforming diagnosis of rare genetic conditions, providing health-economic cost savings and reducing the diagnostic odyssey for affected families. Determination of a precise genetic diagnosis greatly impacts affected families, informing clinical management and enabling prenatal counselling for disease prevention. Despite advances of parallel sequencing, interpretation of splice variants remain a great challenge.

Splicing occurs at the level of pre-mRNA which contains both exon and intron sequences. The spliceosome is a large multimegadalton complex comprised of five small nuclear ribonucleoproteins (U1, U2, U5, and U4/U6), which work synergistically with more than one hundred accessory and regulatory factors to splice together exons into mRNAs encoding protein isoforms. Consensus splice-sites recognised by the splicosome are highly evolutionarily conserved between yeast, plants, Drosophila and vertebrates, with similar constituents and 2-dimensional structures determined for spliceosomal complexes derived from yeast, Drosophila and humans.

Translational tools to predict splicing defects rely on evolutionary conservation of consensus splice-site sequences—and effectively predict adverse consequences of substitutions affecting the essential splice-sites (the almost invariant GT and AG at either end of the intron), which are being recognised increasingly as pathogenic variants in genetic disorders. However, exonic variants creating cryptic splice-sites, and extended splice-site or intronic variants remain challenging to interpret.

There is a need for improvement in prediction of splicing outcomes, and to keep pace with clinical translation of parallel sequencing approaches.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The invention seeks to provide an improvement in prediction of splicing outcomes or otherwise to assist in determining likelihood of Mendelian diseases or cancer and in one embodiment provides a method for determining the likelihood that a genetic variant defines a genetic disease or cancer-associated allele, wherein the genetic variant is located between a genomic sequence encoding a pre-mRNA donor 5′ splice-site and a genomic sequence encoding a related pre-mRNA branch-point site, the related branch-point site being operable with the donor 5′ splice-site in individuals who do not have the genetic disease or cancer to form an intron lariat in pre-mRNA transcribed from the locus, the method comprising:

determining whether a pre-mRNA transcribed from the locus would comprise a sufficient number of nucleotides between the 5′ splice-site and related branch-point site to enable formation of an intron lariat defined by the 5′ splice-site and related branch-point site;

wherein:

where the number of nucleotides between the 5′ splice-site and related branch-point site is insufficient for formation of an intron lariat defined by the 5′ splice-site and related branch-point site, a high likelihood that the genetic variant defines a genetic disease or cancer-associated allele is determined; and

where the number of nucleotides between the 5′ splice-site and related branch-point site is sufficient for formation of an intron lariat defined by the 5′ splice-site and related branch-point site, a low likelihood that the genetic variant defines a genetic disease or cancer-associated allele is determined;

thereby determining the likelihood that the genetic variant of the genetic locus defines a genetic disease or cancer-associated allele.

In another embodiment there is provided a method for treating an individual to minimise the likelihood of development or onset of a genetic disease or cancer, wherein a genetic locus of the individual that controls or is associated with the disease comprises an allele comprising a genetic variant between a genomic sequence encoding a 5′ splice-site and a genomic sequence encoding a related branch-point site, the related branch-point site being operable with the 5′ splice-site in individuals who do not have genetic disease or cancer to form an intron lariat in pre-mRNA transcribed from the locus, the method comprising:

providing or having provided a test sample obtained from an individual for whom likelihood of development or onset of the genetic disease or cancer is to be minimised;

determining or having determined whether a pre-mRNA transcribed from the locus would comprise a sufficient number of nucleotides between the 5′ splice-site and related branch-point site to enable formation of an intron lariat defined by the 5′ splice-site and related branch-point site;

wherein:

where the number of nucleotides between the 5′ splice-site and related branch-point site is insufficient for formation of an intron lariat defined by the 5′ splice-site and related branch-point site, administering a pharmaceutical compound to the individual for treatment of the genetic disease or cancer; and

where the number of nucleotides between the 5′ splice-site and related branch-point site is sufficient for formation of an intron lariat defined by the 5′ splice-site and related branch-point site, not administering a pharmaceutical compound to the individual for treatment of the genetic disease or cancer;

thereby treating the individual for said genetic disease.

Typically the risk of development or onset of genetic disease or cancer in an individual having a genetic variant between a 5′ splice-site and a related branch-point site is lower where an individual having an insufficient number of nucleotides between the 5′ splice-site and branch-point site for formation of an intron lariat in pre-mRNA transcribed from the locus is administered with the compound than it would be if the compound was not administered to the individual, thereby treating the individual for said genetic disease or cancer.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

Figure Legends

FIG. 1. A) Left and Middle: Violin plots representing the genome-wide distribution of exon and intron length in C. elegans (worm), D. melanogaster (flies), D. rerio (fish) and H. sapiens (humans). Right: Histogram showing the relative abundance of small introns <100 nt across species. B) Histogram depicting abundance of human introns <200 nt. Dashed line: 99.9th percentile among all GT-AG human introns. C) Collation of branchpoint analyses described in⁵⁻⁷. Datapoints shown reflect branchpoints identified concordantly by ⅔ studies. Dashed line: 99.9th percentile among all GT-AG human introns. D) i) Family A pedigree. ii) AII:1 with severe scoliosis at age 25. iii) Facial weakness of AII:2 at age 20 years when asked to tightly close eyes. iv) Haematoxylin and eosin (H & E) staining of AII:1 deltoid (biopsy age 15 yrs) shows mild myopathic changes, with occasional internal nuclei (arrow) and evidence for fibre-splitting (asterisks). v) Western blot of skeletal muscle from AII:1 (deltoid, age 15 years) and two controls (C1 and C2, malignant hypothermia negative) shows marked reduction/near absence of normal-sized DOK7 protein. Cl: female, vastus lateralis, 18 years. C2: female, vastus lateralis, 14 years. E) i) Family B pedigree. ii) H & E staining of BII:1 (gastrocnemius) shows marked variation in fibre size, abundant internal nuclei (arrows), and fibre splitting (asterisks). iii) Staining for fast skeletal muscle myosin shows some evidence of fibre type grouping. iv) Western blot of skeletal muscle from BII:1 (deltoid, age 10 years) shows absent normal length emerin. Control 1 (C1): male, malignant hypothermia positive, quadriceps, age 14 years. Control 2 (C2): female, malignant hypothermia negative, quadriceps, age 60 years. Scale bars 200 pm.

FIG. 2. A) Schematic of Family A DOK7 intron 1 deletion, with flanking exons (coloured cylinders), intervening intron sequence, and consensus splicing predictions from Alamut® Visual biosoftware. Blue font: intronic deletion. Lariat branchpoint A is shown in red font. Polypyrimidine tract is italicised. Below: Agarose gels of RT-PCR with adjacent schematic of splicing consequences and effect on encoded DOK7 protein. B) Schematic of Family B EMD intron 5 deletion. Below: Agarose gels of RT-PCR with adjacent schematic of splicing consequences and effect for encoded emerin protein.

FIG. 3. A) Upper: Schematic of EMD genomic locus subcloned into pCMV6, with six numbered exons, and indicative locations of PCR primers used for RT-PCR. Below: WT (79 nt): Intron-5 with sequences deleted in BII:1 shown in blue font. Schematics below depict the specific sequences deleted (shown in grey font) in each expression construct. RC: Reverse complement sequences shown in teal. B) Transfection studies in patient primary myoblasts. Untransfected (UnT) myoblasts used bear a hemizygous exon-6 duplication (c.651_655dupGGGCC) and express low levels of abnormal truncated p.GIn219Argfs*20 emerin and no normal sized emerin protein. Replicate plates from each set of transfections were harvested simultaneously for RT-PCR and western blot. The entire experiment was repeated twice, with identical results. i) RT-PCR of cDNA derived from oligo-dT reverse transcription of mRNA isolated from transfected patient primary myoblasts. Reverse primer 6R2 is positioned at the EMD exon-6 GGGCC duplication, and preferentially amplifies EMD transcripts from the transfected expression construct (optimisation data not shown). ii) Western blot of 10 pg total protein probed with NCL-Emerin and HRP-conjugated secondary antibody. Membranes were reprobed with anti-tubulin as loading control.

FIG. 4. In vitro splicing studies of spliced pre-mRNA products (A) and assembled spliceosome complexes (B). A) Polyacrylamide gel electrophoresis of 32P-labeled COL6A2 pre-mRNAs incubated with a HeLa nuclear extract containing spliceosomal components, for various time periods. Schematics illustrating the nature of the spliced products migrating at different molecular weights are shown. i) WT-COL6A2 and Δ28-COL6A2 pre-mRNAs (with the native exon-9 cryptic 5′SS). The Δ28-COL6A2 deletion within intron-8 renders 5′SS-branchpoint length 36 nt. ii) WTCOL6A2mut and Δ28-COL6A2mut pre-mRNAs with the exon-9 cryptic 5′SS mutated, and, AACOL6A2mut where intron-8 length has been lengthened with polyA nucleotides, restoring 5′SSbranchpoint length to 61 nt. B) Native agarose gel electrophoresis showing temporal progression of spliceosome complex assembly on COL6A2 pre-mRNAs. The migration of A, B, C and B-activated complexes are indicated. i) WT-COL6A2 and Δ28-COL6A2 pre-mRNAs with and without the antisense oligonucleotide masking the exon-9 cryptic 5′SS. ii) Temporal assembly of spliceosomal complexes on WT-COL6A2mut, Δ28-COL6A2mut and AA-COL6A2mut pre-mRNAs. C) Schematic of COL6A2 mini-genes employed in the in vitro splicing assays.

FIG. 5. Pathogenic intronic deletions extracted from ClinVar or LOVD variant databases leading to 5′SS-branchpoint lengths below the hypothesised minimal length. Schematics depict flanking exons (coloured cylinders) and intervening intron sequence. Polypyrimidine tracts are italicised and potential lariat branchpoint A (predicted by Alamut® Visual with scores >50) shown in red font. Reported intronic deletions are in blue font, and splicing outcomes depicted. A) AMN Imerslund-Gräsbeck Syndrome, RT-PCR studies described in¹³. B) COL6A2 Ullrich congenital muscular dystrophy, RT-PCR studies described in²¹. C) DOK7 congenital myasthenic syndrome, a recurrent 15 bp intron-1 deletion identified in ten families^{14,15-18} D) MYH-linked colorectal polyposis (gene also known as MUTYH)37. Two branchpoints with scores of 93.7 and 70.3 were confirmed to be used by the spliceosome in⁵; whereas the third branchpoint shown in grey (score 84.9) was not found in⁵. E) ROGDI Kohlschutter-Tonz syndrome with RT-PCR studies performed in¹⁹. F) MYBPC3 familial hypertrophic cardiomyopathy; one case without a formal classification in ClinVar (RCV000151135.3). Two branchpoints with scores of 57.0 and 50.6 were predicted, with weaker branchpoint shown in grey. The deletion affects the +6 position of the 5′SS; however, compliance with 5′SS-branchpoint minimal length infers strong likelihood for abnormal splicing due to this mechanism.

FIG. 6. A) Pictograms of consensus residues encompassing the 5′ and 3′ splice-sites, subgrouped by intron length. Residues shown include 25 nt intron sequence (annotated as +1, 2, 3 . . . and −1, −2, −3 . . .) plus 5 nt flanking exon (grey shaded regions). B) 3D model of a human spliceosomal B complex formed on a pre-mRNA with a 120 nt intron, determined by cryo-electron microscopy; image adapted from²⁰. RNA helices formed between U6 and intron nucleotides near the 5′SS or between U2 and intron nucleotides adjacent to the branchpoint, are circled. At the 5′ end of the intron, a 17 nt extended helix is formed between the U6 snRNA (via its ACAGAG box and adjacent nucleotides) and intron nucleotides downstream of the 5′SS GU. The branchpoint and upstream nucleotides form a 14 nt helix with the U2 snRNA. Within spliceosomal B complexes assembled on a 120 nt intron, these two extended helices are separated by 15 nm, which corresponds to −21 nt of RNA in an extended conformation²⁰. Thus, minimally 52 intron nucleotides are required to span the 5′SS and the branchpoint in the human B complex without altering its structure.

### DETAILED DESCRIPTION OF THE EMBODIMENTS

Some genetic diseases and cancers present with intron retention, exon skipping or partial exon deletion. The effects of the genetic variants are readily observed in gene expression products in the form of abnormal messenger RNA or abnormal protein isoform(s) that arise from the genetic variants. On the basis of determining the presence of a relevant genetic variant, one is able to determine the likelihood of disease, or to confirm the presence of the disease.

At the time of the invention it was much more difficult to predict or confirm the likelihood of, or presence of disease, on the basis of genomic sequence alone i.e. in the absence of knowledge of whether abnormal messenger RNA or protein isoform(s) are produced, or would be produced from the relevant genomic sequence. The essential question was what is the likelihood that an individual will develop a particular genetic disease or cancer given a particular genomic sequence of the individual? That question has been difficult to answer.

While genetic variants at functional residues of a consensus 5′ splice-site, 3′ splice-site, branch-point site and some regulatory elements had been shown to associate with some diseases, these genetic variants were understood not to account for many of the forms of disease or cancer that had been observed. Indeed some of the genetic variants had been observed not to give rise to disease at all.

Further complicating an answer was that a very large number of genetic variants of intronic and exonic sequences had been identified. Some of these variants had been observed as purine/pyrimide, pyrimidine/purine, purine/purine or pyrimidine/pyrimidine substitutions, with or without an insertion or deletion event.

It was appreciated that some of these variants might be relevant to disease. However, the functional significance of the bulk of identified genetic variants was not known. In this context, these genetic variants had been classified as ‘variants of uncertain significance’ (VUS). A VUS is a variant for which there is insufficient evidence (as defined by the American College for Medical Genetics and Genomics²²; ACMG) doi:10.1038/gim.2015.30, to allow unequivocal determination of the relationship of the variant with genetic disease or cancer. A methodology enabling one to assess which VUS were risk factors for disease was required.

In the studies described herein, the inventor has determined that those VUS that result in a shortened intragenic distance between genomic sequences encoding the 5′ splice-site and downstream, relevant branch-point site are genetic variants that are more likely than not to be associated with disease, and in particular, more likely than not to result in abnormal pre-mRNA splicing including, but not limited to, intron retention, exon skipping or partial exon deletion. On this basis the inventor has developed a methodology which enables one to address the relevance of at least some genetic variants or VUS to genetic disease and cancer, enabling one to predict the likelihood, or confirm the existence of genetic disease or cancer.

The methodology is particularly advantageous insofar as it enables one to further classify at least some of a bewildering number of variants for which there is insufficient evidence of causal relationship to disease or cancer, or as risk factors for disease or cancer.

While not wanting to be bound by hypothesis, it is considered that a shortening of the intragenic distance between a 5′ splice-site and branch-point site below a critical threshold measure provides insufficient space for a U1/U2-dependent spliceosome complex to assemble on pre-mRNA transcribed from the relevant locus to exert splicing function, especially to form and/or excise the intron lariat. This may result in the spliceosome complex inappropriately utilising an alternate 5′ splice-site, or branch-point site, or 3′splice-site (i.e. “alternate” in the sense of being a splice-site or branch-point site not typically utilised in normal pre-mRNA processing with respect to the relevant mRNA isoform(s) linked to a genetic condition), resulting in exon skipping, partial exon deletion or intron retention in the abnormal pre-mRNA splicing event(s).

As described herein, any of the above splicing events may result in: 1) an abnormal mRNA transcript with genetic information removed (exon-skipping, partial exon deletion), 2) an abnormal mRNA transcript with ectopic genetic information included (intron retention, use of an alternate splice-site), 3) Multiple abnormal mRNA transcripts with either genetic information abnormally removed or inserted. As amino-acids are encoded by three DNA bases, insertion or deletion of genetic information disrupts the amino-acid reading frame in two-thirds of instances. A common consequence of disruption of the amino-acid reading frame is the abnormal encoding of a stop signal (TAA, TGA, TAG). All abnormal mRNA transcripts are likely to produce a non-functional or dysfunctional protein (due to insertion or removal of amino acids or truncation of a protein due to an abnormal stop signal), thereby providing an association with, or causation of the relevant genetic disease or cancer. Additionally, abnormal mRNA transcripts can be degraded by a mechanism called nonsense-mediated decay that will result in no, or very low levels, of translated protein product.

As exemplified herein, the inventors have found that the genetic variants cause abnormal mRNA transcripts by using recombinant engineering techniques that reduce the intragenic distance below the critical threshold required for the spliceosome to assemble. The inventors have also found that the genetic variants are not found in respect of a construct that has been engineered to increase the intragenic distance between the donor site and branch-point so as to exceed the threshold. These studies demonstrate the importance of the intragenic distance between the 5′ splice-site and the related branch-point site.

In one embodiment there is provided a method for determining the likelihood that a genetic variant of a genetic locus defines a genetic disease or cancer-associated allele, wherein the genetic variant is located between a genomic sequence encoding a pre-mRNA 5′ splice-site and a genomic sequence encoding a related pre-mRNA branch-point site, the related branch-point site being operable with the 5′ splice-site in individuals who do not have the genetic disease or cancer to form an intron lariat from pre-mRNA transcribed from the locus, the method comprising:

determining whether a pre-mRNA transcribed from the locus would comprise a sufficient number of nucleotides between the 5′ splice-site and related branch-point site to enable formation of an intron lariat defined by the 5′ splice-site and related branch-point site;

wherein:

where the number of nucleotides between the 5′ splice-site and related branch-point site is insufficient for formation of an intron lariat defined by the 5′ splice-site and related branch-point site, a high likelihood that the genetic variant defines a genetic disease or cancer-associated allele is determined; and

where the number of nucleotides between the 5′ splice-site and related branch-point site is sufficient for formation of an intron lariat defined by the 5′ splice-site and related branch-point site, a low likelihood that the genetic variant defines a genetic disease or cancer-associated allele is determined;

thereby determining the likelihood that the genetic variant of the genetic locus defines a genetic disease or cancer-associated allele.

In another embodiment there is provided a method for determining the likelihood that a genetic variant of a genetic locus defines a genetic disease or cancer-associated allele, wherein the genetic variant is located between a genomic sequence encoding a pre-mRNA 5′ splice-site and a genomic sequence encoding a related pre-mRNA branch-point site, the related branch-point site being operable with the 5′ splice-site in individuals who do not have the genetic disease or cancer to form an intron lariat from pre-mRNA transcribed from the locus, the method comprising:

determining whether a pre-mRNA transcribed from the locus would comprise a sufficient number of nucleotides between the 5′ splice-site and the related branch-point site to enable a spliceosome A- complex, when bound to the pre-mRNA at the region of the 5′ splice-site and related branch-point site, to transition to a B-complex or C-complex;

wherein:

where the number of nucleotides between the 5′ splice-site and related branch-point is insufficient to enable a spliceosome A-complex to transition to a B-complex or a C-complex, a high likelihood that the genetic variant is a genetic disease or cancer-associated allele is determined; and

where the number of nucleotides between the 5′ splice-site and related branch-point is sufficient to enable a spliceosome A-complex to transition to a B-complex or a C-complex, a low likelihood that the genetic variant is a genetic disease or cancer-associated allele is determined;

thereby determining the likelihood that the genetic variant of the genetic locus defines a genetic disease or cancer-associated allele.

In a further embodiment there is provided a method for determining the likelihood that a genetic variant of a genetic locus defines a genetic disease or cancer-associated allele, wherein the genetic variant is located between a genomic sequence encoding a pre-mRNA 5′ splice-site and a genomic sequence encoding a related pre-mRNA branch-point site, the related branch-point site being operable with the 5′ splice-site in individuals who do not have the genetic disease or cancer to form an intron lariat from pre-mRNA transcribed from the locus, the method comprising:

determining whether a pre-mRNA transcribed from the locus would comprise a sufficient number of nucleotides between the 5′ splice-site and the related branch-point site to enable a spliceosome complex to cleave the pre-mRNA at the 5′ splice-site and related branch-point site;

wherein:

where the number of nucleotides between the 5′ splice-site and related branch-point is insufficient to enable a spliceosome complex to cleave the pre-mRNA at the 5′ splice-site and/or related branch-point site, a high likelihood that the genetic variant is a genetic disease or cancer-associated allele is determined; and

where the number of nucleotides between the 5′ splice-site and related branch-point is sufficient to enable a spliceosome complex to cleave the pre-mRNA at the 5′ splice-site and related branch-point site, a low likelihood that the genetic variant is a genetic disease or cancer-associated allele is determined;

thereby determining the likelihood that the genetic variant of the genetic locus defines a genetic disease or cancer-associated allele.

In a yet further embodiment there is provided a method for determining the likelihood that a genetic variant of a genetic locus defines a genetic disease or cancer-associated allele, wherein the genetic variant is located between a genomic sequence encoding a pre-mRNA 5′ splice-site and a genomic sequence encoding a related pre-mRNA branch-point site, the related branch-point site being operable with the 5′ splice-site in individuals who do not have the genetic disease or cancer to form an intron lariat from pre-mRNA transcribed from the locus, the method comprising:

determining whether a pre-mRNA transcribed from the locus would comprise greater than 45-57, preferably greater than 47 to 52 nucleotides, preferably greater than 47 nucleotides between the 5′ splice-site and related branch-point site;

wherein:

where the intragenic distance would comprise greater than 45-57, preferably greater than 47 to 52 nucleotides, preferably greater than 47 nucleotides a low likelihood that the genetic variant is a disease-associated allele is determined; and

where the intragenic distance would comprise less than 45-57, preferably less than 47 to 52 nucleotides, preferably less than 47 nucleotides a high likelihood that the genetic variant is a disease-associated allele is determined;

thereby determining the likelihood that the genetic variant of the genetic locus defines a genetic disease or cancer-associated allele.

In the above described embodiments, a sample (herein test sample) may be provided from an individual in whom the genetic variant has been detected, and for whom the risk of genetic disease or cancer is to be determined. Typically, the genetic variant may be a variant of uncertain significance (VUS). A VUS is a variant for which there is insufficient evidence (as defined by the American College for Medical Genetics and Genomics²²; ACMG) doi:10.1038/gim.2015.30, to allow unequivocal determination of the relationship of the variant with genetic disease or cancer.

In a preliminary step, a test sample comprising genomic DNA provided from an individual in whom risk for genetic disease or cancer is to be determined may be sequenced by gene sequencing methods routinely used in the art and exemplified in the Examples herein.

The genomic sequence obtained is assessed to determine whether the genetic variant is located between a genomic sequence encoding a 5′ splice-site recognised by a U1/U2-dependent spliceosome and a related branch-point site, also recognised by a U1/U2-dependent spliceosome. A related branch-point site refers to an adenosine residue that may be utilised by a U1/U2-dependent spliceosome together with the 5′ splice-site for intron lariat formation and subsequent splicing out of the relevant intron.

A consensus genomic sequence encoding a 5′ splice-site recognised or cleaved by a U1/U2-dependent spliceosome can be determined by standard techniques, including those exemplified in the Examples herein. A consensus genomic sequence encoding a branch-point site recognised or cleaved by a U1/U2-dependent spliceosome can be determined by standard techniques, including those exemplified in the Examples here. See also Reference 5 herein.

The assessment of the genomic sequence may be done in the context of comparison with a control. A suitable control may be genetic information in relation to the relevant genomic sequence of the genetic locus obtained from individuals who do not have genetic disease or cancer. Exemplary reference human genome sequences include the “Genome Reference Consortium Build 37” also referred to as “hg19” (World Wide Web at ncbi.nlm.nih.gov/assembly/GCF_000001405.13), or the Genome Reference Consortium Human Build 38 patch release 12 (GRCh38.p12) (World Wide Web at ncbi.nlm.nih.gov/assembly/GCF_000001405.38), or any sequenced human genome from an individual or individuals not exhibiting or carrying a Genetic disorder, such as sequence information within the Genome Aggregation Database (gnomAD; https://gnomad.broadinstitute.org/; 2019 release encompassing 125,748 exome sequences and 15,708 whole-genome sequences from individuals without a severe paediatric Mendelian

For example, the genomic sequence obtained from the test sample may be compared with the control to determine or confirm the presence and/or location of the genetic variant in relation to a normal genetic locus. Such a comparison may be useful to determine or confirm the amount of shortening of the intragenic distance between the consensus genomic sequences for the splice donor and related branch-point sites, and to consider whether there are further branch-point, splice donor, or splice acceptor sites that could be utilised as a consequence of the genetic variant, potentially resulting in intron retention, exon splicing or partial exon deletion, as described further herein.

Typically, the genetic variant for which association with genetic disease or cancer is to be determined does not involve the genomic sequence that encodes the pre-mRNA sequence that forms the 5′ splice-site or related branch-point site. The canonical genomic sequence that encodes a slice donor site cleavable by a U1/U2-dependent spliceosome is 5′-AGGT-3′, with cleavage occurring 3′ to AG in that sequence. Accordingly, the genetic variant does not involve substitution of adenosine, guanine or thymine in the canonical sequence, more preferably does not involve substitution of guanine in the canonical sequence. Accordingly, the pre-mRNA transcribed from the locus may be cleaved at the 5′ splice-site, provided that there is sufficient number of nucleotides between the 5′ splice-site and the related branch-point site. The canonical genomic sequence that encodes a branch-point site cleavable by a U1/U2-dependent spliceosome is adenosine followed by a polypyrimidine rich tract of about 15 nucleotides, with cleave occurring 5′ adjacent the adenosine. Accordingly, the genetic variant does not involve substitution of adenosine in the canonical sequence. Accordingly, the pre-mRNA transcribed from the locus may be cleaved at the branch-point site, provided that there is sufficient number of nucleotides between the 5′ splice-site and related branch-point site. Thus in one embodiment, the 5′ splice-site or respective branch-point site is not comprised in the genetic variant.

In the above described embodiments, the genetic variant may result in a shortening of the intragenic distance between the genomic sequence encoding the pre-mRNA 5′ splice-site and the genomic sequence encoding the related pre-mRNA branch-point site 1 to 5000 nucleotides, preferably 1 to 500 nucleotides, more preferably 1 to 50 nucleotides, as compared with the distance between the genomic sequence encoding the pre-mRNA 5′ splice-site and the genomic sequence encoding the related pre-mRNA branch-point site in an individual who does not have the relevant genetic disease or cancer. As exemplified herein, deletion events resulting in a shortening of 10 to 25 nucleotides results in mutation in the form of intron retention, exon splicing or partial exon deletion. In one embodiment, the genetic variant comprises a deletion of a sequence of nucleotides, preferably a sequence of from 1 to 50, more preferably 1 to 25, more preferably 1 to 10 nucleotides.

It will be understood that in some circumstances, a shortening of the intragenic distance may involve a deletion event, a further mutation event such as a nucleotide insertion, a nucleotide sequence insertion and/or nucleotide substitution.

Typically, genomic sequence is assessed to determine whether a pre-mRNA transcribed from the locus would comprises a sufficient number of nucleotides between the 5′ splice-site and related branch-point site to enable formation of an intron lariat defined by the 5′ splice-site and related branch-point site; or a sufficient number of nucleotides between the 5′ splice-site and the related branch-point site to enable a spliceosome A-complex, when bound to the pre-mRNA at the region at the 5′ splice-site and related branch-point site, to transition to a B-complex or C-complex; or greater than 45-57, preferably greater than 47 to 52 nucleotides, preferably greater than 47 nucleotides between the 5′ splice-site and related branch-point site; or a sufficient number of nucleotides between the 5′ splice-site and related branch-point site to enable formation of an intron lariat defined by the 5′ splice-site and related branch-point site.

In a particularly preferred embodiment, there is provided a method for determining the likelihood that a genetic variant of a genetic locus defines a genetic disease or cancer-associated allele, wherein the genetic variant is located between a genomic sequence encoding a pre-mRNA 5′ splice-site and a genomic sequence encoding a related pre-mRNA branch-point site, the splice donor and branch-point sites cleavable by U1/U2-dependent spliceosome, the related branch-point site being operable with the 5′ splice-site in individuals who do not the genetic disease or cancer to form an intron lariat from pre-mRNA transcribed from the locus, the method comprising:

determining whether a pre-mRNA transcribed from the locus would comprise greater than 47 nucleotides between the 5′ splice-site and related branch-point site;

wherein:

where the intragenic distance would comprise greater than 47 nucleotides a low likelihood that the genetic variant is a disease-associated allele is determined; and

where the intragenic distance would comprise less than 47 nucleotides a high likelihood that the genetic variant is a disease-associated allele is determined;

thereby determining the likelihood that the genetic variant of the genetic locus defines a genetic disease or cancer- associated allele.

As described herein, the inventor has recognised that a genetic variant between the genomic sequence for the pre-mRNA 5′ splice-site and the genomic sequence for the pre-mRNA related branch-point site may lead to intron retention where the splicesome cannot excise an intron lariat because the splice donor and branch-point sites are located too close together. This leads to a failure to correctly splice out the relevant intron. Thus the methods described herein relate to determining the likelihood that a genetic variant of a genetic locus produces an RNA molecule comprising an intron retention event, wherein the genetic variant defines an intragenic distance from a genomic sequence encoding a pre-mRNA 5′ splice-site to a genomic sequence encoding a pre-mRNA related branch-point site of 47 nucleotides or less, the related branch site being operable with the 5′ splice-site in individuals who do not produce RNA from the locus having the intron retention event. The method comprises the steps of:

determining whether a pre-mRNA transcribed from the locus would comprise a further branch-point site in an intronic sequence that is 3′ adjacent to the related branch-point site that could be utilised by a U1/U2-dependent spliceosome complex to form a lariat defined by the 5′ splice-site and the further branch-point site;

wherein a determination that a pre-mRNA transcribed from the locus would comprise the further branch-point site determines a low likelihood that the genetic variant would produce an RNA molecule comprising an intron retention event in the form of retention of an intron comprising the related branch-point site;

wherein a determination that a pre-mRNA transcribed from the locus would not comprise the further branch-point site determines a high likelihood that the genetic variant would produce an RNA molecule comprising an intron retention event in the form of retention of an intron comprising the related branch-point site;

thereby determining the likelihood of the genetic variant producing an RNA molecule comprising an intron retention event.

The inventor has demonstrated that exon skipping may arise where the intragenic shortening between the splice donor and branch-point sites and the promiscuity of the U1/U2-dependent spliceosome complex results in the spliceosome utilising a further branch-point site in an intron that is 3′ to the intron in which the related branch-point site is located. The result is the formation of a lariat that includes the exon located 3′ to the intron in which the related branch-point site is located. Thus the methods described herein relate to determining the likelihood that a genetic variant of a genetic locus produces an RNA molecule comprising an exon skipping event, wherein the genetic variant defines an intragenic distance from a genomic sequence encoding a pre-mRNA 5′ splice-site to a genomic sequence encoding a pre-mRNA related branch-point site of 47 nucleotides or less, the related branch site being operable with the 5′ splice-site in individuals who do not produce RNA from the locus having the exon skipping event. The method comprises the steps of:

determining whether a pre-mRNA transcribed from the locus would comprise a further branch-point site in an intronic sequence that is 3′ adjacent to the related branch-point site that could be utilised by a U1/U2-dependent spliceosome complex to form a lariat defined by the 5′ splice-site and the further branch-point site;

wherein a determination that a pre-mRNA transcribed from the locus would comprise the further branch-point site determines a high likelihood that the genetic variant would produce an RNA molecule comprising an exon skipping event in the form of skipping of an exon that is 3′ adjacent to the related branch-point site;

wherein a determination that a pre-mRNA transcribed from the locus would not comprise the further branch-point site determines a low likelihood that the genetic variant would produce an RNA molecule comprising the exon skipping event;

thereby determining the likelihood of the genetic variant producing an RNA molecule comprising an exon skipping event.

The inventor has demonstrated that a partial exon deletion may arise where the intragenic shortening between the splice donor and branch-point sites results in the U1/U2-dependent spliceosome complex utilising a further donor splice site in an exon located 5′ to the donor splice site. This mutation results in the splicing out of exon sequence between the further 5′ splice-site and the 5′ splice-sites. Thus the methods described herein relate to determining the likelihood that a genetic variant of a genetic locus produces an RNA molecule comprising a partial exon deletion event, wherein the genetic variant defines an intragenic distance from a genomic sequence encoding a pre-mRNA 5′ splice-site to a genomic sequence encoding a pre-mRNA related branch-point site of 47 nucleotides or less, the related branch site being operable with the 5′ splice-site in individuals who do not produce RNA from the locus having the partial exon deletion event. The method comprises the steps of:

determining whether a pre-mRNA transcribed from the locus would comprise a further donor splice site in an exon sequence that is 5′ adjacent to the donor splice site that could be utilised by a U1/U2-dependent spliceosome complex to form a lariat defined by the further 5′ splice-site and the branch-point site;

wherein a determination that a pre-mRNA transcribed from the locus would comprise the further 5′ splice-site determines a high likelihood that the genetic variant would produce an RNA molecule comprising a partial exon deletion event in the form of deletion of exon sequence between the further 5′ splice-site and 5′ splice-site;

wherein a determination that a pre-mRNA transcribed from the locus would not comprise the further 5′ splice-site determines a low likelihood that the genetic variant would produce an RNA molecule comprising the partial exon deletion event;

thereby determining the likelihood of the genetic variant producing an RNA molecule comprising an partial exon deletion event.

The inventor has also demonstrated that partial exon deletion may arise where the intragenic shortening between splice donor and related branch-point sites results in a U1/U2-dependent spliceosome complex utilising both a further branch-point site and a further splice acceptor site (otherwise known as 3′ splice site), the further splice acceptor site being located in an exon located 3′ to the splice acceptor site that is utilised by the spliceosome complex in individuals that do not produce mRNA comprising the partial exon deletion event. This mutation results in the splicing out of exon sequence between the spice acceptor site and the further splice acceptor site. Thus the methods described herein relate to determining the likelihood that a genetic variant of a genetic locus produces an RNA molecule comprising a partial exon deletion event, wherein the genetic variant defines an intragenic distance from a genomic sequence encoding a pre-mRNA 5′ splice-site to a genomic sequence encoding a pre-mRNA related branch-point site of 47 nucleotides or less, the related branch site being operable with the 5′ splice-site in individuals who do not produce RNA from the locus having the partial exon deletion event. The method comprises the steps of:

determining whether a pre-mRNA transcribed from the locus would comprise a further acceptor splice site in an exon sequence that is 3′ adjacent to the acceptor splice site that could be cleaved by a U1/U2-dependent spliceosome complex;

wherein a determination that a pre-mRNA transcribed from the locus would comprise the further splice acceptor site determines a high likelihood that the genetic variant would produce an RNA molecule comprising a partial exon deletion event in the form of deletion of exon sequence between the splice acceptor site and the further splice acceptor site;

wherein a determination that a pre-mRNA transcribed from the locus would not comprise the further splice acceptor site determines a low likelihood that the genetic variant would produce an RNA molecule comprising the partial exon deletion event;

thereby determining the likelihood of the genetic variant producing an RNA molecule comprising an partial exon deletion event.

In certain embodiments, the individual the subject of the assessment or treatment methods described above may be asymptomatic. In other embodiments, the individual may have some but not all symptoms of a relevant condition or cancer.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Summary:

Background: Splice variants are a common cause of human genetic disorders, though challenging to identify. Abnormal splicing can be devastating for the encoded protein, inducing a frame-shift or in-frame deletion/insertion of multiple residues. There is great need for improved informatics pipelines to detect and predict splice-altering variants.

Methods & results: Genomic sequencing identified intronic deletions in EMD (intron-5 reduced from 79 to 56 nucleotides, nt) or DOK7 (intron-1 reduced from 76 to 66 nt), sparing all consensus splice-sites, in two index families with neuromuscular disorders. Normal splicing was abolished in muscle biopsies, with associated deficiency of emerin or DOK7 protein by western blot. The mechanistic basis for abnormal splicing is due to biophysical constraint, whereby the human U1/U2 spliceosomal machinery is unable to assemble within critically shortened introns, stalling in A complexes. Restoration of 5′ splice-site to branchpoint (5′SS-branchpoint) length with non-specific sequences restores spliceosome assembly and normal splicing, excluding primary influence of intronic splice enhancers. Incremental restoration of EMD intron-5 length defines 45-47 nt as a critical minimal distance between 5′SS-branchpoint for spliceosome assembly; aligning closely with the observed minimal 5′SS-branchpoint distance among human introns (49 nt). We identify 23 further families with pathogenic intronic deletions due to the minimal 5′SS-branchpoint length mechanism, in 10 genes across different fields of genomic medicine.

Conclusions: Intronic deletions that unnaturally shorten 5′SS-branchpoint minimal length present a novel class of splice variant, not factored by splicing algorithms, and relevant to diagnosis and precision medicine across the breadth of Mendelian disorders and cancer genomics.

### Results

### Intron Length in Humans and Model Organisms

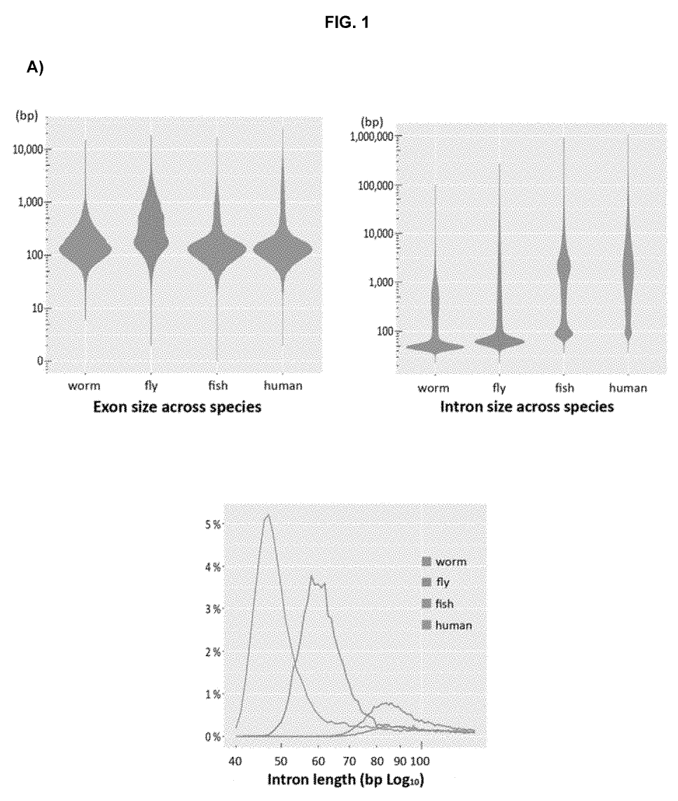

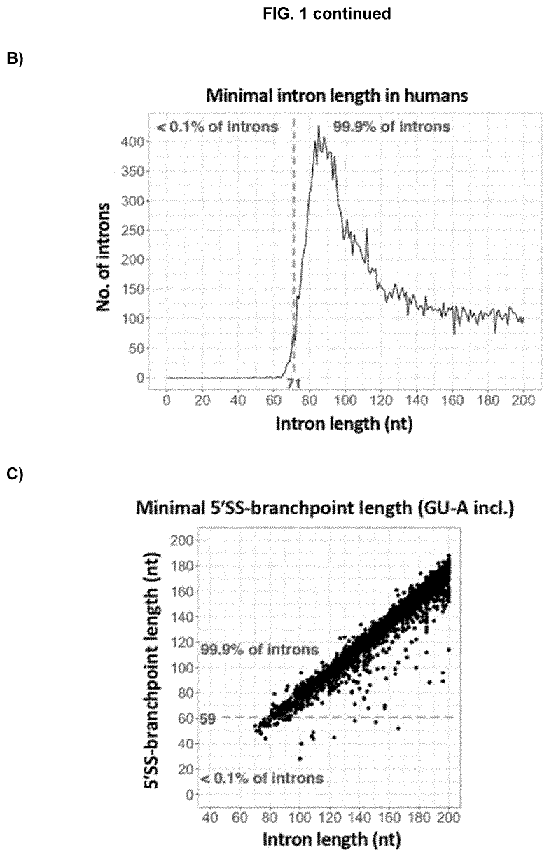

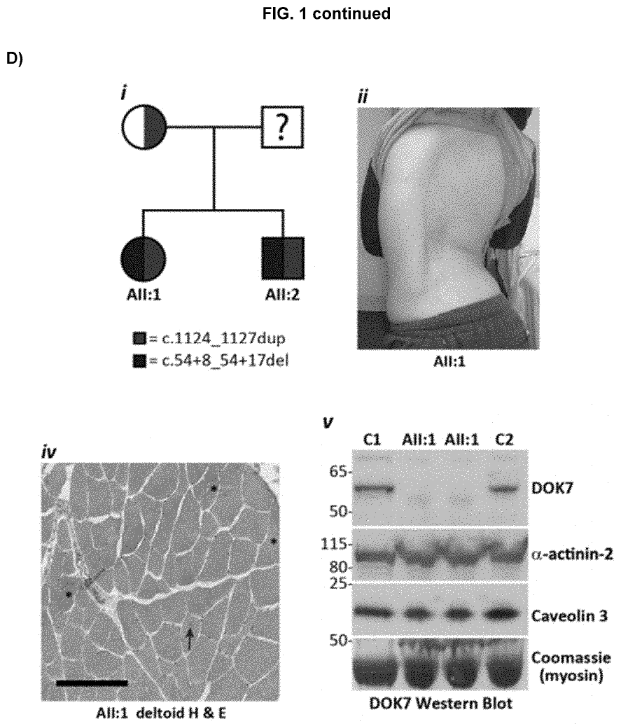

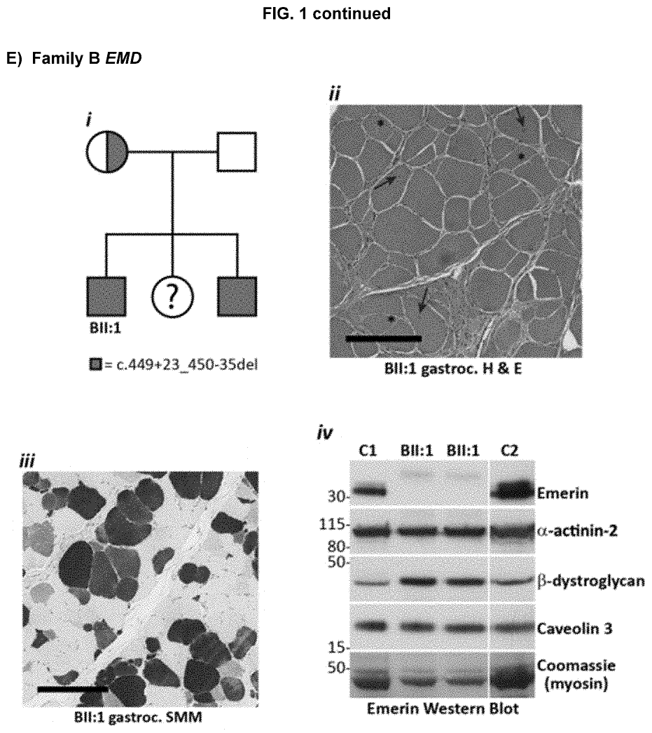

There are important differences in intron features between humans and model organisms. Human introns show great diversity in length, with a median length of 1,455 nucleotides (nt; 90th percentile 148-11,098) (FIG. 1A). In contrast, Drosophila introns are generally shorter, with a median length of 72 nt (90th percentile 56-2,374 nt) and C. elegans introns even shorter, with a median length of 63 nt (90th percentile 45-764 nt). In particular, short introns <71 nt are extremely rare in the human genome (0.1th percentile, FIG. 1B), though represent the majority of introns of Drosophila and C. elegans (FIG. 1A). Among the shortest human introns (<200 nt), there is a direct correlation between intron length, and the distance between the 5′ splice-site and branchpoint (5′SS-branchpoint). The shorter the intron, the shorter the 5′SS-branchpoint distance; with three genome-side branchpoint studies defining an abrupt minimum 5′SS-branchpoint distance threshold of 49 nt, with 59 nt the 0.1th percentile (FIG. 1C). Herein we demonstrate that intronic deletions that unnaturally shorten 5′SS-branchpoint length below a critical, minimum length, present a novel class of pathogenic splice variant currently overlooked by informatics pipelines.

### Patient Clinical and Genetic Findings

Family A: Two affected siblings with a clinical history of fluctuating, severe limb-girdle muscle weakness, born at term to non-consanguineous, Caucasian parents (FIG. 1D). Both siblings presented in the neonatal period, becoming floppy and weak after vaccination, requiring hospitalization for the female proband (at age 2 months). Both siblings showed persistent fluctuations in muscle strength throughout infancy and early childhood, with facial weakness delayed motor milestones, but no speech delay or intellectual impairment. The female sibling (AII:1) required BiPAP (bilevel positive airway pressure) aged 8 years for respiratory weakness and suffered from recurrent infections. She lost ambulation aged 10 years and required scoliosis surgery at 15 years. Muscle biopsy at age 15 years showed mild myopathic changes (FIG. 1Div). Creatine kinase (CK) levels were normal to mildly elevated (37 and 700 U/L; normal <200 U/L). On re-examination at age 25 years, she had bilateral mild ptosis, fatigable limb-girdle weakness, severe scoliosis (FIG. 1Dii) and severe restrictive ventilatory defect. Electrocardiogram and echocardiogram were normal. The male sibling (AII:2) developed lumbar lordosis aged 4 years, required use of a power wheelchair at 8 years and scoliosis surgery at 14 years. At 18 years, he required BiPAP for respiratory weakness, with respiratory function tests aged 20 years revealing severe reduction in forced vital capacity of 2.25 litres; 45% of predicted. Serum CK levels and echocardiogram were normal.

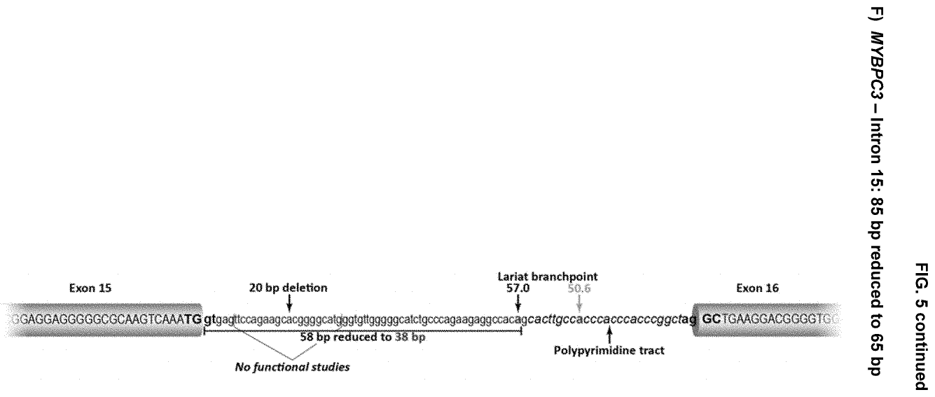

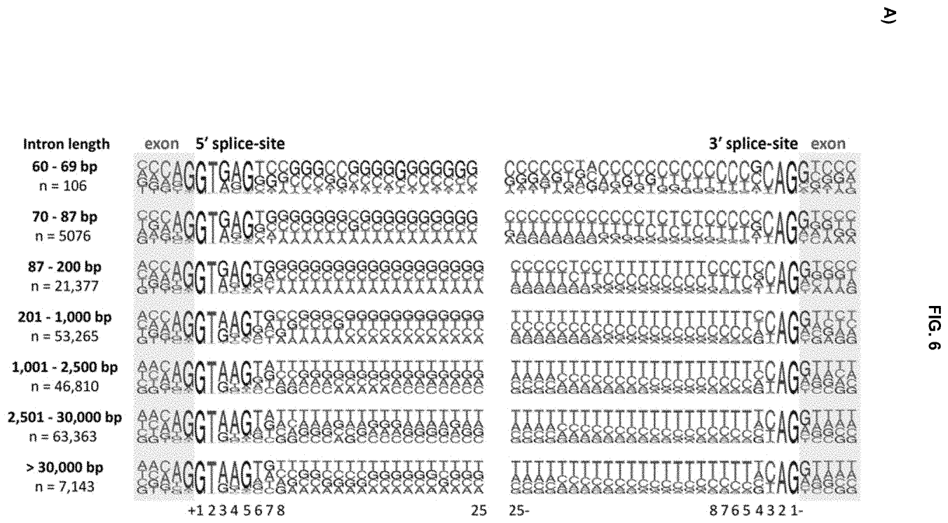

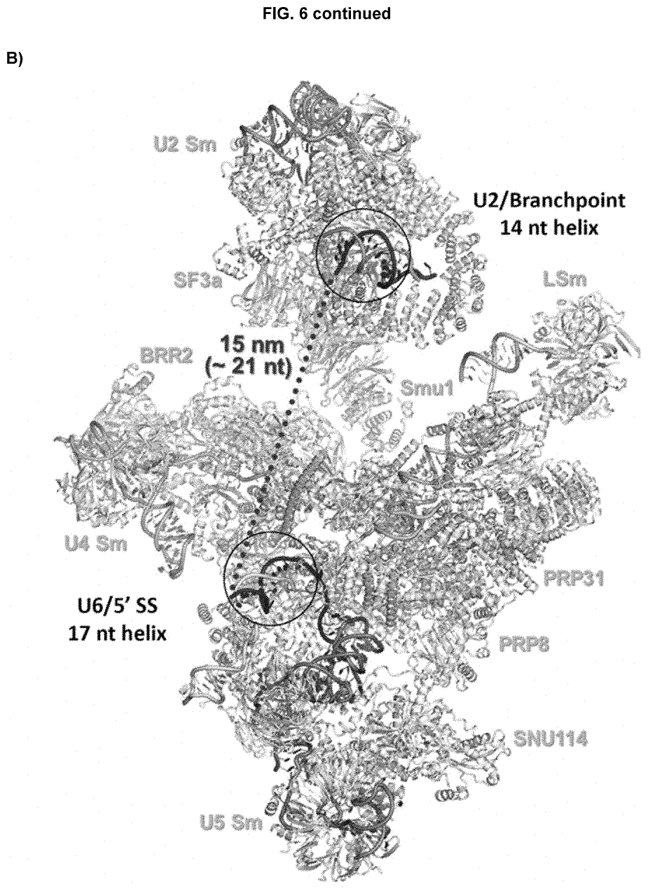

Whole exome sequencing performed for AII:1 and AII:2 revealed compound heterozygous DOK7 variants (FIGS. 1Di and 2A); a gene associated with congenital myasthenic syndrome (CMS). The maternal allele carried the common DOK7 exon-6 duplication (GRCh37/hg19 chr4:3494837_3494840dupTGCC), reported in >140 recessive CMS cases in the Leiden Open Variant (LOVD) database. Both siblings also carried a novel 10 base-pair (bp) deletion within DOK7 intron-1 (GRCh37:chr4:3465164_3465173del), at the +8 position of the extended splice-site, and a position without significant base preference (see FIG. 6A). This variant was not present in gnomAD, exome variant server (EVS), ClinVar or LOVD databases. Paternal DNA was unavailable to confirm inheritance. The intron-1 deletion was not predicted to cause abnormal splicing of the DOK7 pre-mRNA using Alamut® Visual (Interactive Biosoftware, Rouen, France) that incorporates five splicing algorithms (SpliceSiteFinder-Like, MaxEntScan, GeneSplicer, Human Splicing Finder and NNSPLICE).

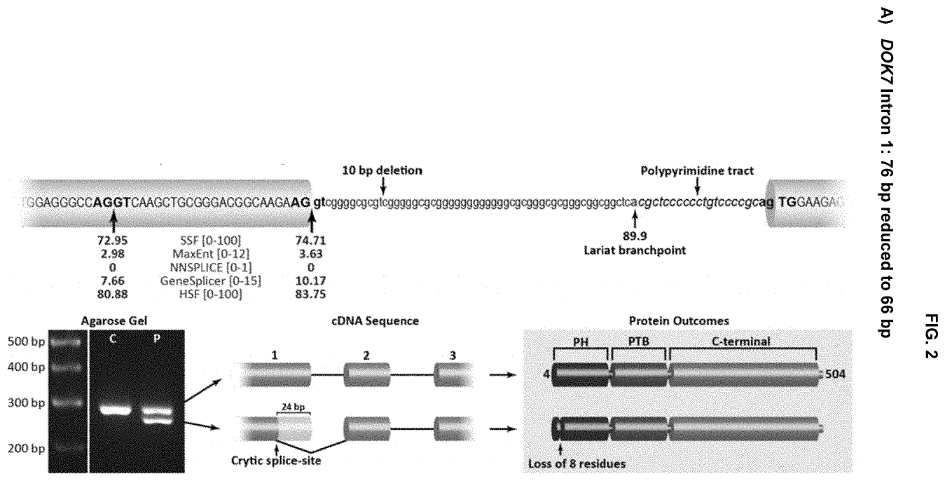

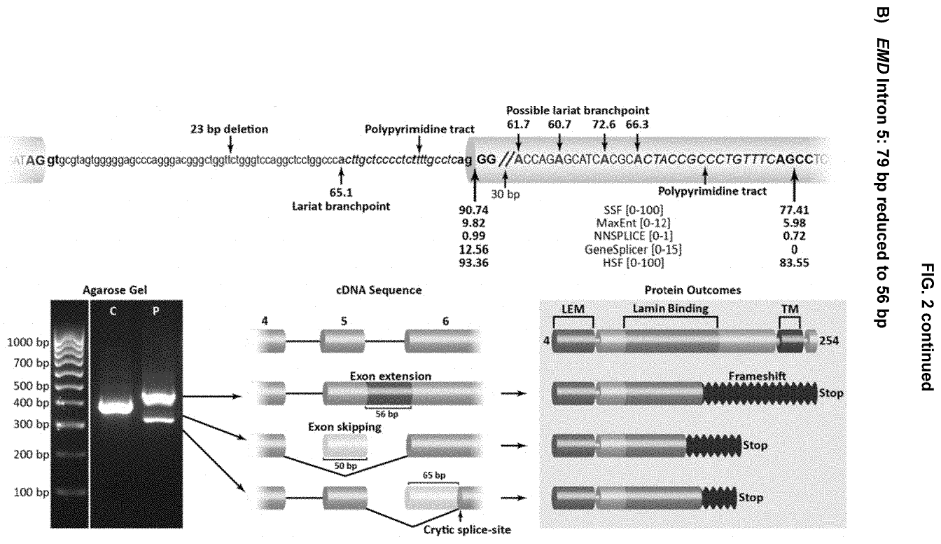

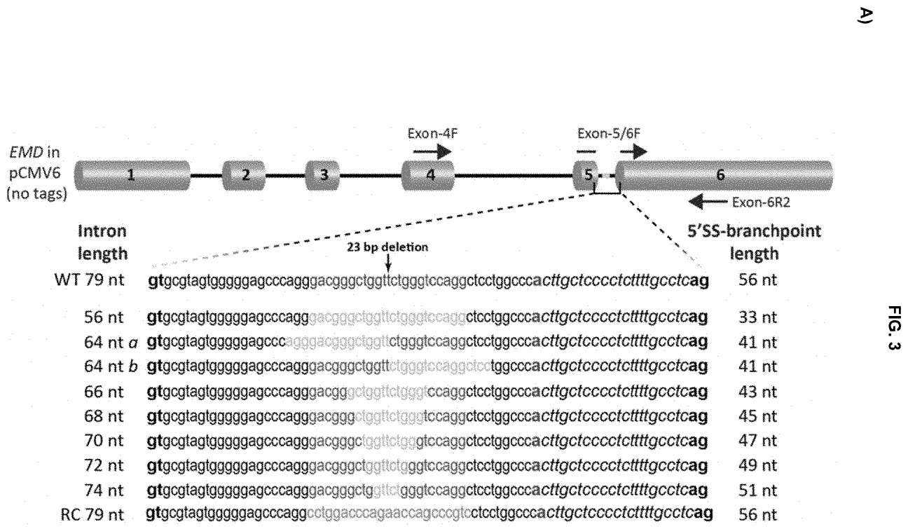

Family B: BII:1 was born at term to non-consanguineous parents with no family history of neuromuscular disease (FIG. 1E). He presented aged 3 years with distal lower limb weakness, and required surgery for ankle contractures at 9 years. Examination at 9 years showed scapulo-peroneal muscle weakness with bilateral scapular winging and reduced muscle bulk for his age. Muscle biopsy aged 10 years showed marked variation in fibre size with fibre splitting and abundant internal nuclei (FIG. 1Eii). Serum CK levels were mildly elevated (585 U/L). Neuromuscular gene panel screening revealed a novel hem izygous 23 bp deletion within intron-5 of EMD (at the +23 position, GRCh37:chrX:153609185_153609207de1), a gene associated with X-linked Emery-Dreifuss muscular dystrophy (FIG. 1E and 2B). The hemizygous deletion was maternally inherited and not predicted to cause abnormal splicing using Alamut® Visual software. This variant was not present in gnomAD, EVS, LOVD or

ClinVar databases.

### Small Intronic Deletions Ablate Normal Splicing of DOK7 and EMD Genes

Reverse transcription PCR studies (RT-PCR) of mRNA extracted from skeletal muscle from affected individuals AII:1 (DOK7) and BII:1 (EMD) showed clear evidence for pathogenic splicing abnormalities (FIG. 2A and 2B).

For AII:1, a cDNA amplicon encompassing exons 1-3 of DOK7 showed two bands (FIG. 2A).

Sanger sequencing confirmed the upper band represents normal splicing, whereas the lower band represents an abnormally spliced mRNA utilizing an exon-1 cryptic 5′ splice-site (5′SS); derived only from the paternal allele with the 10 bp intron-1 deletion (determined using an informative SNP in exon-1). Use of the exon-1 cryptic 5′SS removes 24 nt from the DOK7 mRNA and loss of eight conserved residues within the encoded DOK7 pleckstrin homology domain (FIG. 2A). Western blot analyses of skeletal muscle biospecimens show marked reduction/near deficiency of DOK7 protein in AII:1, relative to aged-matched controls (FIG. 1Dv).

RT-PCR of cDNA derived from BII:I amplifying EMD exons 3-6 revealed absence of normallyspliced mRNA (FIG. 2B). Sanger sequencing of amplicons showed the EMD intron-5 hem izygous 23 nt deletion primarily induced exon-5 extension or use of a cryptic 3′ splice-site (3′SS) within exon-6, with exon-5 skipping a minor species (asterisk, FIG. 2B). Each abnormally spliced EMD transcript induces a frameshift to the emerin reading frame, resulting in C-terminal missense amino acids and a premature stop codon. Encoded mutant forms of emerin have an abnormal lam inbinding domain and lack a transmembrane anchor. Western blot analyses confirmed deficiency of normal-sized emerin protein in muscle of the affected proband BII:1 (FIG. 1Eiv).

Despite confirming abnormal splicing of DOK7 and EMD in the muscle biospecimens in Family A and Family B, the exact cause was not clear, and therefore we extensively investigated the mechanistic basis for abnormal splicing.

EMD Partial Splicing is Enabled with 5′SS-Branchpoint Length of 47 nt.

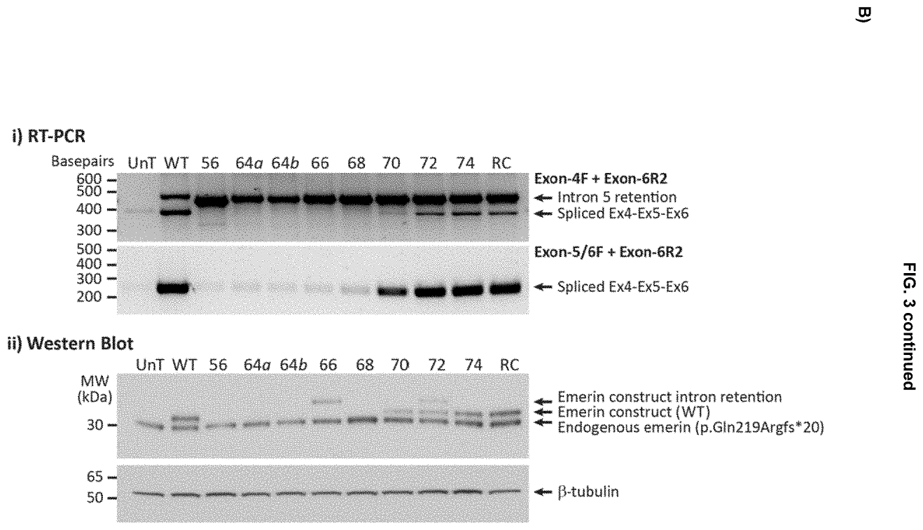

We derived a panel of EMD full gene expression constructs, manipulating intron-5 length, in the context of an obligate lariat branchpoint adenine (FIG. 3A, branchpoint A shown in red font). mRNA derived from the wild-type (WT) EMD construct is spliced (FIG. 3Bi, lower band) and translated into full length emerin protein (FIG. 3Bii, middle band); readily distinguished from the abnormal truncated emerin expressed endogenously in the EMD patient myoblasts utilised for this study (see legend; FIG. 3Bii, untransfected (UnT), lower band). However, significant levels of intron-5 retention are also observed by RT-PCR with transfection of the WT EMD construct (FIG. 3Bi, dominant upper band); inferring inherent challenges splicing the 79 nt intron-5 when overexpressed.

Nevertheless, recapitulating the 23 nt deletion in B11:1 ablates normal splicing of EMD (FIG. 3Bi, Lane 56 nt), with concordant absence of full length emerin on western blot (FIG. 3Bii, Lane 56 nt). Incremental restoration of EMD intron-5 length shows abrupt (partial) restoration of splicing and emerin protein production with a 5′SS-branchpoint length of 47 nt (when intron-5 =70 nt, FIG. 3B, Lane 70 nt). Higher migrating abnormal emerin protein detected with intron-5 lengths of 66 and 72 nt likely correspond to proteins translated from EMD mRNA where intron-5 retention is in-frame (FIG. 3Bii, upper bands).

Normal splicing of an EMD construct with a reverse complement 23 nt sequence substituted for residues deleted in B11:1 (FIG. 3B, Lane RC) argue against loss of intronic regulatory elements as a primary basis for abnormal splicing (though may be contributory). Further, two distinct 15 nt deletions within intron-5 (FIG. 3B, Lanes 64a and 64b), which render a 5′SS-branchpoint length of 41 nt, are both unable to be spliced—arguing that ‘minimal length’, rather than loss of a specific intronic motif, is the more likely causal basis for splicing abnormalities.

### The Human Spliceosome is Unable to Assemble Within, or Splice, Critically Shortened Introns

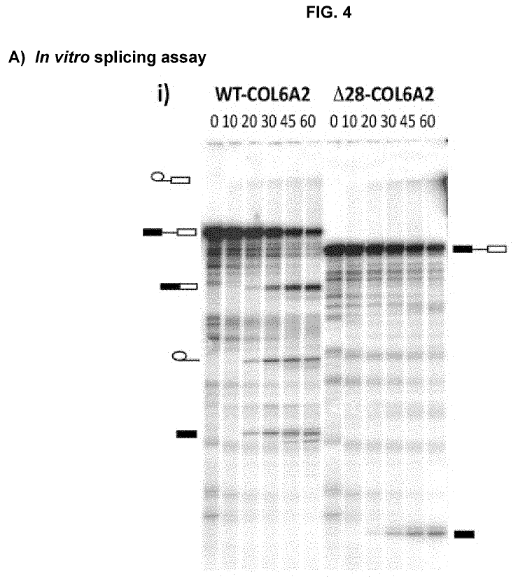

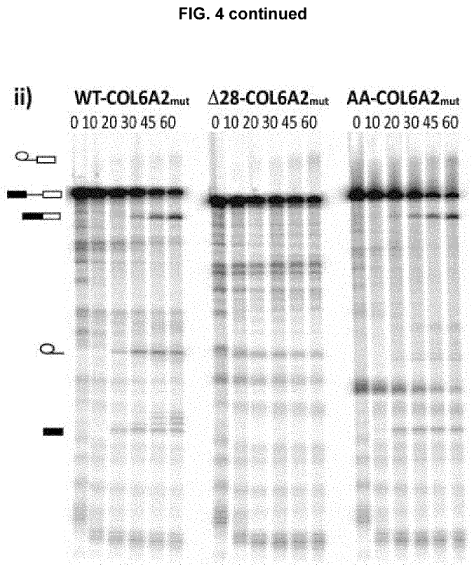

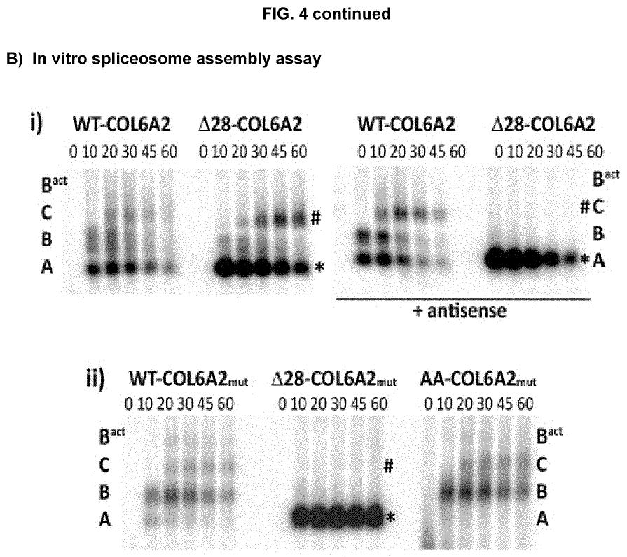

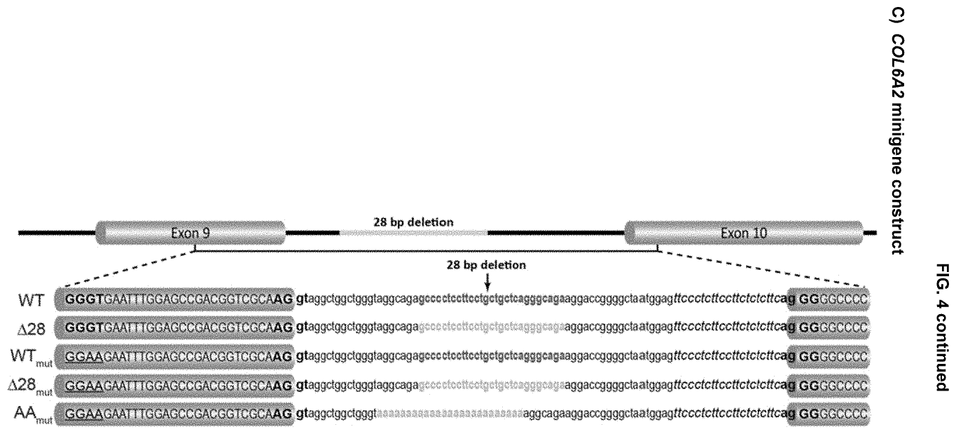

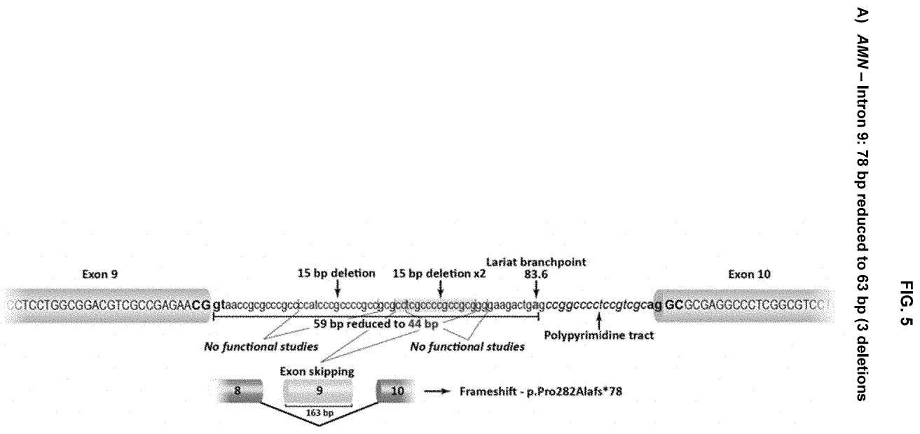

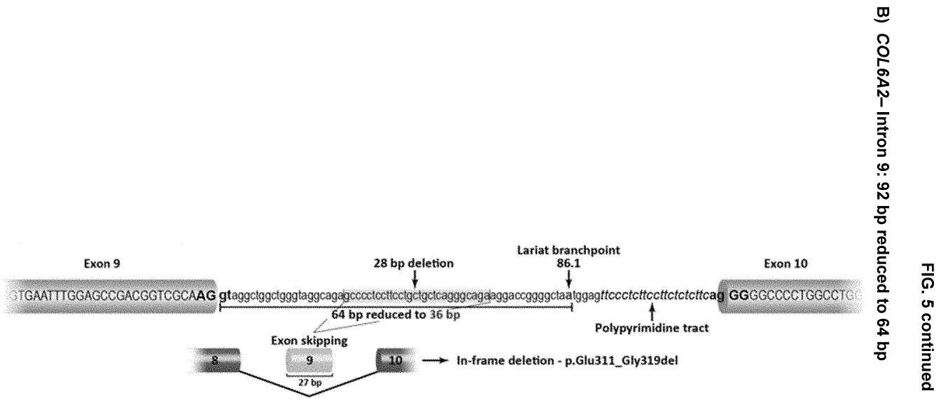

In vitro splicing assays were used to confirm whether biophysical constraint precluding spliceosome assembly is the underlying mechanistic basis for abnormal splicing. Modeling a previously reported 28 nt deletion in COL6A2 intron-9, splicing and excision of the intron-9 lariat occurs efficiently for a wildtype (WT) COL6A2 pre-mRNA (exons 9-10), but fails for the Δ28-COL6A2 pre-mRNA (FIG. 4Ai); with spliceosome assembly stalling in A complexes that bridge 5′SS and branchpoint (FIG. 4Bi, asterisk). As shown in FIG. 4Ai, deletion of 28 nt results in the formation of an abnormal 5′exon 56 nt cleavage product for the Δ28-COL6A2 pre-mRNA (lower right, black rectangle). This appears due to abnormal spliceosome assembly on a weak cryptic 5′SS in exon-9, 23 nt upstream of the natural 5′SS at the exon-9/intron junction (see FIG. 4C); as masking the exon-9 cryptic 5′SS with an antisense DNA oligonucleotide potently blocks C complex assembly (FIG. 4Bi, hash). Despite detectable C complex assembly for Δ28 using the cryptic splice 5′SS (FIG. 4Bi, hash), the spliceosome appears unable to execute excision of an intron lariat (no detectable excised splicing product, FIG. 4Ai).

Mutation of the cryptic 5′SS site prevents is use by the spliceosome, and results in normal spliceosome assembly and splicing for the WTₘᵤₜ pre-mRNA (FIG. 4Aii and 4Bii). In contrast, there is no observed splicing of Δ28ₘᵤₜ (and concomitant absence of the abnormal 56 nt cleavage product; FIG. 4Aii, middle), with spliceosome assembly stalled in A complexes (FIG. 4Bii, middle, asterisk). Restoring Δ28ₘᵤₜ intron-9 5′SS-branchpoint length to 61 nt with a non-specific poly A sequence (AAₘᵤₜ; intron-9 length 89 nt) restores normal splicing (FIG. 4Aii, right) and temporal progression of spliceosome assembly (FIG. 4Bii, right). These data provide compelling evidence that failed spliceosome assembly is due primarily to distance, rather than loss of intronic enhancer elements.

ClinVar Data-Mining Identifies 23 Additional Families with Pathogenicity Likely Due to Minimal 5′SS-Branchpoint Deletions

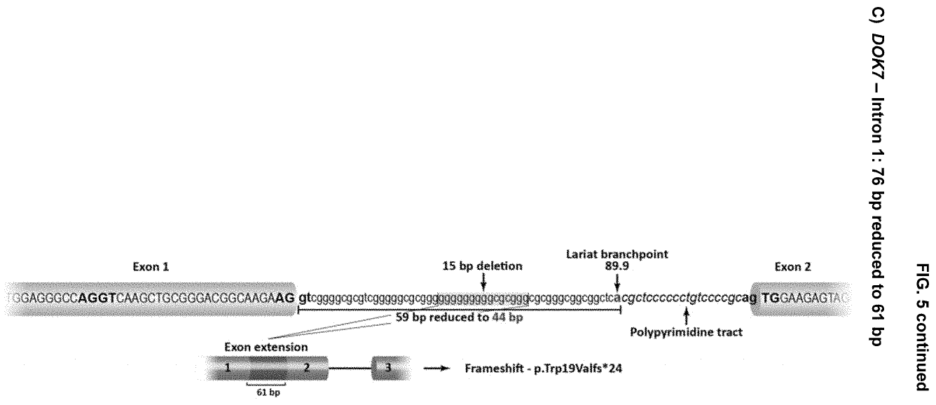

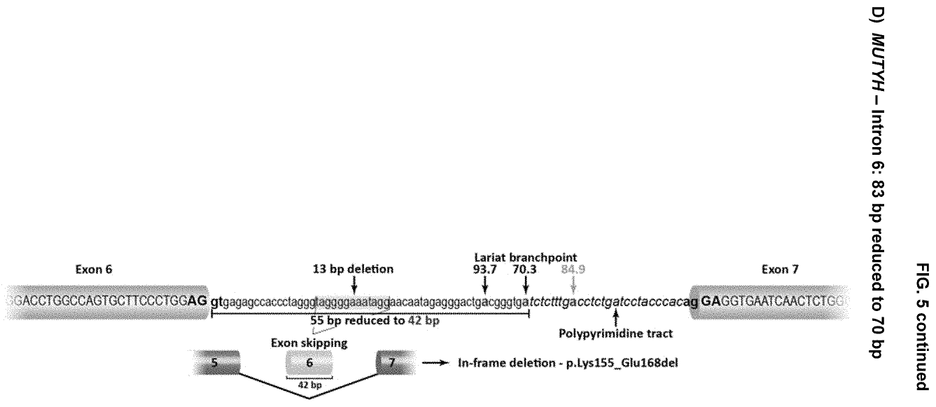

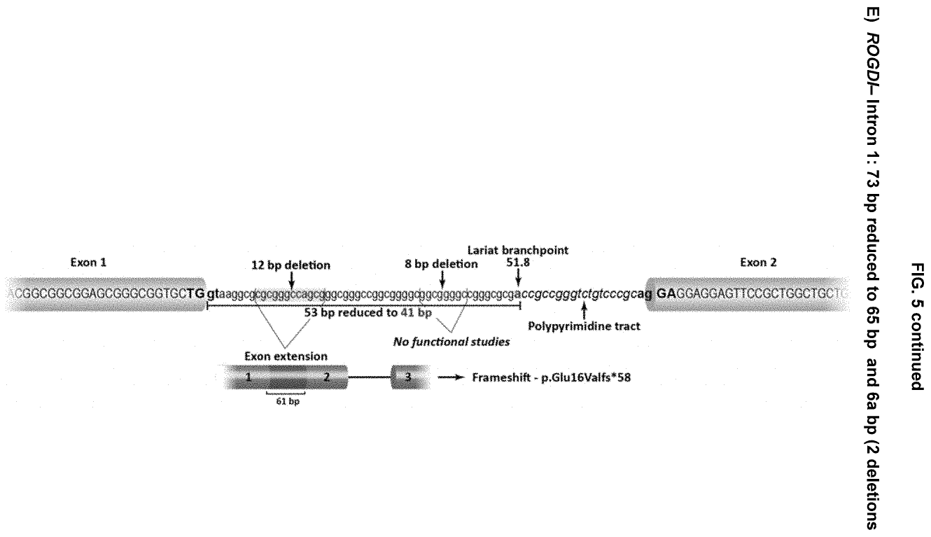

We performed informatics analyses of intronic deletions submitted to ClinVar or the Leiden Open Variant database (LOVD), and identified ten deletions classified as pathogenic or likely pathogenic, sparing consensus extended splice-sites, which reduced predicted 5′SS-branchpoint length to less than 47 nt (FIG. 5); 10 families with DOK7 congenital myasthenia, 3 families with ROGDI Kohlschutter-Tonz syndrome, 7 families with AMN Imerslund-Gräsbeck Syndrome, 2 families with MYH-linked colorectal polyposis (gene also known as MUTYH) and 1 family with COL6A2 Ulrich congenital muscular dystrophy. Despite no adverse consequences predicted by splicing algorithms, due to phenotypic fit and clinical suspicion, splicing analyses were performed for variants affecting 21/23 families. In all 21 cases, aberrant splicing was confirmed to be associated with the intronic deletions. We further identified an intronic deletion identified in one case of suspected MYBPC3 familial hypertrophic cardiomyopathy (RCV000151135.3, not formally classified), that reduce 5′SS-branchpoint length below 47 nt, and likely to be abnormally spliced due to this mechanism (FIG. 5F).

### Confirmed DOK7 Congenital Myasthenia Supports Salbutamol Intervention for Family A and Prenatal Counseling for Family B

Since salbutamol treatment is known to be beneficial for DOK7 patients, salbutamol treatment was initiated and titrated to 6 mg twice a day in both siblings from Family A. Proband AII:1, dependent on her motorized wheelchair over the last 15 years, after 6 months salbutamol treatment could walk 40 meters independently and ˜100 meters with a guided frame. On examination she showed reduced dysphonia, ptosis had improved, and lung infections were less frequent. Following salbutamol treatment, Patient AII:2 could stand without using his hands, and walk a few steps. He could mobilize with a guided frame for 40 meters and was able to drive a car. Transfers between the chair, bed and the shower became more feasible and he recently managed to climb a flight of stairs.

Family B, with two affected children, now have a confirmed genetic diagnosis that has enabled prenatal genetic counseling.

### Discussion

The ten pathogenic 5′SS-branchpoint deletions we collate herein, affect introns with canonical U1/U2 splice-sites. Extensive curation of three genome-wide studies of human branchpoints identifies only 6 canonical GU-AG introns with plausible 5′SS-branchpoints of <50 nt (FIG. 1C); this extreme rarity speaks to atypical or specialist splicing. FIG. 6A shows the shortest human introns (60-87 nt) have several features distinct from ‘typical introns’ (201-2,500 nt); a G-C gradient from splice 5′SS to 3′SS, preference for G>A at +3 position and C rather than T preference within the polypyrimidine tract. These features, and potentially other exonic or intronic motifs or structural features unique to short introns and their flanking exons, may recruit specialised splicing co-factors to aide splicing of short introns.

FIG. 3 establishes that while an EMD pre-mRNA with a 5′SS-branchpoint distance of 47 nt is able to be spliced, though inefficiently; a 5′SS-branchpoint length of 45 nt could not be spliced. Further, our in vitro splicing studies using a patient-based COL6A2 Δ28 pre-mRNA indicates the spliceosome appears unable to transition from A to B complexes when assembling within a critically shortened intron (FIG. 4B); inferring that a minimal 5′SS-branchpoint distance is required for efficient spliceosomal B complex formation. The critical distance between the 5′SS and branchpoint is determined by the 3-dimensional space between two helices formed within the spliceosomal B complex (see FIG. 6B, helices circled). At the 5′ end of the intron, a 17 nt extended helix is formed between U6 (via its ACAGAG box and adjacent nucleotides) and intronic nucleotides downstream of the 5′SS GU, while the branchpoint and upstream nucleotides form a 14 nt helix with the U2 snRNA. For B complexes formed on a premRNA with a 120 nt intron, these two extended helices are separated by 15 nm (see FIG. 6B), which corresponds to ˜21 nt of RNA in an extended conformation. Extrapolation of these three measurements (17 nt intron/U6 helix+14 nt intron/U2 helix+21 nt span between helices) therefore identifies 52 intronic nucleotides as the minimal span between the 5′SS and branchpoint to encompass and bridge these two helices, without altering the structure of the spliceosome.

However, slightly shorter lengths between 5′SS and branchpoint may be tolerated for B complex assembly (as observed for EMD @ 47 nt in FIG. 3), via minimal movement of the head domain with respect to the main body of the B complex, and/or, if the U6/intron helix were shortened by a few base pairs.

We advocate scrutiny of any deletion in a phenotypically consistent gene that renders overall intron length <71 nt (0.1th percentile among human introns), or 5′SS-branchpoint length <59 nt (0.1th percentile among human introns); with our data alerting extreme risk for splicing abnormalities for introns with 5′SS-branchpoint length reduced to <50 nt.

In summary, we define critical shortening of 5′SS-branchpoint minimal length as a novel mechanistic basis and primary determinant for abnormal splicing in human genetic conditions.

Genomics informatics pipelines currently overlook non-coding intronic deletions. Only short introns <100 nt may be interrogated by exome sequencing pipelines, which capture ˜50 nt of the flanking intron. However, whole genome sequencing informatics pipelines enable genome wide screening for potential 5′SS-branchpoint deletions, which must also be considered in the context of structural rearrangements. The 5′SS-branchpoint minimal length mechanism is relevant to all human introns bearing canonical splice-sites that recruit the U1/U2 spliceosome (>99% of all introns), and thus relevant across the breadth of Mendelian disorders and cancer genomics.

### Methods

### Ethics and Consent

Ethical approval was obtained from the Human Research Ethics Committees of the Children's Hospital at Westmead, Australia (10/CHW/45) with written, informed consent from all participants.

Parallel Sequencing

Parallel sequencing of known neuromuscular disease genes was performed by a commercial gene panel (v2) offered by PathWest Laboratory, Australia. WES^{1,2} and RNA sequencing (RNA-seq)³ was performed by the Broad Institute of MIT and Harvard University, USA, as described previously.

Exon/Intron Species Data

RefSeq browser extensible data (BED) files representing exon/intron regions were obtained from the UCSC table browser (https://genome.ucsc.edu/cgi-bin/hgTables). Intron/exon datasets were further filtered by selecting one transcript (per gene) possessing the largest length and number of exons. Data (and scripts) are hosted at https://github.com/kidsneuro-lab/minimal_introns.

Analysis of 5′SS-Branchpoint Length Among Human Introns with Canonical Splice-Sites

Human introns were extracted from NCBI reference sequences⁴. Introns less than 70 nt in length (n=121) were curated manually, identifying 32 introns <66 nt length. 20/32 introns <66 nt in length were determined unlikely to be true introns and excluded, due to the following reasons: 1) Crossreferencing annotated exon/intron boundaries between GRCh37 and GRCh38 genome assemblies and RNA-seq data from ENCODE provided convincing evidence for mis-annotation/mis-alignment of sequences; 2) Lack of evidence within RNA-seq data from ENCODE and introns lacked identifiable splice-sites; 3) Lack of consensus between ENSEMBL and RefSeq. Manual curation data is available at https://github.com/kidsneuro-lab/minimal_introns. Branchpoint datasets from⁵⁻⁷ were combined and filtered to include only GT-AG introns (n=181,139). Data-points presented in FIG. 1C present a high-confidence dataset of branchpoints defined as being concordantly identified in ⅔ studies (n=39,628).

### EMD Expression Construct

A pCMV6-entry vector containing the EMD genomic locus GRCh37:chrX:153607583_153609881 was purchased from BlueHeron Biotech. The native EMD stop codon precedes the vector epitope tags which are therefore not encoded within the EMD pre-mRNA. The EMD genomic sequence ordered had two synonymous substitutions; GRCh37:chrX:153609413G>T and GRCh37:chrX:153609416T>G, introducing a unique BspEl restriction site for molecular manipulation of EMD intron-5. Gene fragments (gBlocks) with the sequences described in FIG. 3A were supplied by Integrated DNA technologies and subcloned into pCMV6-EMD via Pstl and BspEl restriction digest. Constructs were verified by Sanger sequencing.

### Primary Myoblast Transfection

EMD primary myoblasts derived from a male proband with a pathogenic 5 nt duplication in EMD intron-6 (GRCh37:chrX:153609443_153609447dupGGGCC) were transfected with Lipofectamine 3000 reagent, according to the manufacturer's instructions. Cells were harvested 72 hours following transfection for western blot and RT-PCR.

RNA Isolation, cDNA Synthesis and RT-PCR

RNA isolation was performed from 30×8 pm thick muscle cryosections (10 mm2 surface area) or from 20 cm2 surface area of transfected primary myoblasts using Invitrogen TRIzol® Reagent according to the product user guide. RNA was purified using the RNeasy® Mini Kit from QIAGEN, according to the kit protocol. cDNA was synthesized from 1 pg of total skeletal muscle RNA using oligo-dT and/or random hexamers using the Invitrogen SuperScript™ IV First-Strand Synthesis System as per the manufacturer's protocol. DOK7 RT-PCR used primers; 5′UTR-F1 5′-CGCGGAACCATGACAGAAG-3′ or 5′UTR-F2 5′-TTTTGAAAGTGACCCTGGGC-3′ with exon-3R 5′-TGGGACAGGCAGACAATGG-3′. EMD RT-PCR used primers: exon-3F 5′-CTTCCCAAGAAAGAGGACGC-3′ and exon-6R1 5′-GTGAGCCATGAAGAGGAAGATG-3′; exon-6R2 5′-CCTGGCGATCCTGGCCCA-3′ (primer preferentially amplifying EMD cDNA derived from the construct); exon-5/6F 5′-GAGTGCAAGGATAGGGAACG-3′ (bridging primer specific for normally-spliced EMD pre-m RNA).

### Western Blot

Western blot of skeletal muscle and transfected myoblasts was carried out as described previously⁸. Primary antibodies used were anti-DOK7 (AF6398@1:1000, R&D systems), NCLEmerin (1:1000) and NCL-β-DG (1:250) from Leica Biosystems, Caveolin-3 (610421@1:1000, BD Transduction Laboratories), anti-actinin-2 (4A3@1:250,000; kind gift from A. Beggs, Children's Hospital Boston, USA) and α-beta tubulin @ 1:5000 (clone E7, Developmental Studies Hybridoma bank). α-mouse light chain HRP conjugated (1:5000) and a-rabbit light chain HRP conjugated (1:3000) secondary antibodies were used followed by detection with ECL chemiluminescent reagents (GE healthcare).

### Human Intron Splice-Site Pictograms

BEDTools (http://bedtools.readthedocs.io/en/latest/) and hg19 fasta sequences (http://hgdownload.cse.ucsc.edu/goldenpath/hg19/chromosomes/) were used to extract sequences for all human introns (25 nt intron +5 nt flanking exon). Splice-site consensus sequences were visualized using BioPerl's pictogram module (http://search.cpan.org/dist/BioPerl/Bio/Draw/Pictogram.pm). Truncated fasta files and pictograms are hosted at https://github.com/kidsneuro-lab/minimal_introns.

Extraction of Submissions Involving Intronic Deletions from ClinVar and LOVD

ClinVar variants where the molecular consequence contained “intron”, and variant was denoted as a “del/indel”, were extracted from a transformed set of ClinVar variants (https://github.com/macarthur-lab/clinvar)⁹. ClinVar variants were cross-referenced with UCSC to refine a short-list of confirmed intronic variants for manual curation.⁹. Leiden Open Variant Database (LOVD) (http://www.dmd.nl/) variants were extracted using their application programming interface (API) then cross-referenced with the HGVS python module (https://github.com/biocommons/hgvs)¹⁰ to obtain genomic coordinates. ClinVar and LOVD variants were cross-referenced with UCSC to refine a short-list of confirmed intronic variants for manual curation.

### In Vitro Spliceosome Assembly and Splicing Studies

Splicing reactions contained 40% (v/v) HeLa nuclear extract prepared according to¹¹, with 65 mM KCl, 3 mM MgCl2, 2 mM ATP, 20 mM creatine phosphate and 10 nM 32P-labelled, m7G-capped COL6A2 pre-mRNA, incubated at 30° C. for the indicated times. Uniformly³²P-labeled, m⁷G(5′)ppp(5′)G-capped pre-mRNA was synthesized in vitro by incorporation of [³²P]UTP (3000 Ci/mmol; Perkin Elmer) in a T7 runoff transcription. The antisense DNA oligonucleotide used to block the cryptic 5′SS (5′-CCAAATTCACCCTGTGTAGG-3′) was added at 1 pM final concentration to the splicing reaction. Spliceosomal complexes were analyzed on 2% native agarose gels¹² after adding heparin (final concentration of 0.1 pg/pl). RNA was recovered at the indicated time points by PCI extraction, ethanol precipitated and analyzed on a 10% polyacrylamide gel containing 6 M urea. Unspliced pre-mRNA, splicing intermediates and products were detected using a Typhoon phosphoimager (GE Healthcare).

WT-COL6A2 and Δ28-COL6A2 DNA sequences used for in vitro splicing reaction were synthesized by BlueHeron Biotech (USA) and amplified by PCR with the primers: COL6A-T7-F1 5′-ACCTAATACGACTCACTATAgggtgcccatgatgctttgagg-3′ and COL6A-R1 5′-atgcctctgtgagaccagtcc-3′. COL6A-T7-F1 comprises a T7 promoter. PCR products were gel purified and used as template for in vitro transcription reactions. WT-COL6A2mut, Δ28-COL6A2mut, AACOL6A2mut constructs, inserted in Puc18, were synthesized by GenScript Inc. (USA). Each construct contained an upstream T7 promoter and downstream Kpnl restriction site. Vectors were linearized with the Kpnl restriction enzyme, gel purified and used as template in in vitro transcription reaction.

### Abbreviations

API, application programming interface; BED, browser extensible data; bp, basepairs; CK, creatine kinase; EVS, exome variant server; H & E, haematoxylin and eosin; LOVD, Leiden open variant database; nt, nucleotide; RNA-seq, RNA sequencing; UCSC, University of California Santa Cruz; VUS, variants of uncertain significance; WES, whole exome sequencing.

### REFERENCES

1. Ghaoui R, Cooper S T, Lek M, et al. Use of Whole-Exome Sequencing for Diagnosis of Limb-Girdle Muscular Dystrophy: Outcomes and Lessons Learned. JAMA Neurol 2015;72:1424-32.
2. O'Grady G L, Lek M, Lamande S R, et al. Diagnosis and etiology of congenital muscular dystrophy: We are halfway there. Ann Neurol 2016;80:101-11.
3. Cummings B B, Marshall J L, Tukiainen T, et al. Improving genetic diagnosis in Mendelian disease with transcriptome sequencing. Sci Transl Med 2017;9.
4. O'Leary N A, Wright M W, Brister J R, et al. Reference sequence (RefSeq) database at NCBI: current status, taxonomic expansion, and functional annotation. Nucleic Acids Res 2016;44:D733-45.
5. Mercer T R, Clark M B, Andersen S B, et al. Genome-wide discovery of human splicing branchpoints. Genome Res 2015;25:290-303.
6. Pineda J M B, Bradley R K. Most human introns are recognized via multiple and tissuespecific branchpoints. Genes Dev 2018;32:577-91.
7 Taggart A J, Lin C L, Shrestha B, Heintzelman C, Kim S, Fairbrother W G. Large-scale analysis of branchpoint usage across species and cell lines. Genome Res2017;27:639-49.
8. Cooper S T, Lo H P, North K N. Single section Western blot: improving the molecular diagnosis of the muscular dystrophies. Neurology 2003;61:93-7.
9. Zhang X, Minikel E V, O'Donnell-Luria A H, MacArthur D G, Ware J S, Weisburd B. ClinVar data parsing. Wellcome Open Res 2017;2:33.
10. Hart R K, Rico R, Hare E, Garcia J, Westbrook J, Fusaro V A. A Python package for parsing, validating, mapping and formatting sequence variants using HGVS nomenclature. Bioinformatics 2015;31:268-70.
11. Dignam J D, Lebovitz R M, Roeder R G. Accurate transcription initiation by RNA polymerase II in a soluble extract from isolated mammalian nuclei. Nucleic Acids Res 1983;11:1475-89.
12. Behzadnia N, Hartmuth K, Will C L, Luhrmann R. Functional spliceosomal A complexes can be assembled in vitro in the absence of a penta-snRNP. RNA 2006;12:1738-46.
13. S. M. Tanner, A. C. Sturm, E. C. Baack, S. Liyanarachchi, A. de la Chapelle, Inherited cobalamin malabsorption. Mutations in three genes reveal functional and ethnic patterns. Orphanet J Rare Dis 7, 56 (2012).
14. D. Selcen, M. Milone, X. M. Shen, C. M. Harper, A. A. Stans, E. D. Wieben, A. G. Engel, Dok-7 myasthenia: phenotypic and molecular genetic studies in 16 patients. Ann Neurol 64, 71-87 (2008).
15. A. Ben Ammar, F. Petit, N. Alexandri, K. Gaudon, S. Bauche, A. Rouche, D. Gras, E. Fournier, J. Koenig, T. Stojkovic, A. Lacour, P. Petiot, F. Zagnoli, L. Viollet, N. Pellegrini, D. Orlikowski, L. Lazaro, X. Ferrer, G. Stoltenburg, M. Paturneau-Jouas, F. Hentati, M. Fardeau, D. Sternberg, D. Hantai, P. Richard, B. Eymard, Phenotype genotype analysis in 15 patients presenting a congenital myasthenic syndrome due to mutations in DOK7. J Neurol 257, 754-766 (2010).
16. J. Cossins, W. W. Liu, K. Belaya, S. Maxwell, M. Oldridge, T. Lester, S. Robb, D. Beeson, The spectrum of mutations that underlie the neuromuscular junction synaptopathy in DOK7 congenital myasthenic syndrome. Hum Mol Genet 21, 3765-3775 (2012).
17. D. Lashley, J. Palace, S. Jayawant, S. Robb, D. Beeson, Ephedrine treatment in congenital myasthenic syndrome due to mutations in DOK7. Neurology 74, 1517-1523 (2010).
18. U. Schara, N. Barisic, M. Deschauer, C. Lindberg, V. Straub, N. Strigl-Pill, M. Wendt, A. Abicht, J. S. Muller, H. Lochmuller, Ephedrine therapy in eight patients with congenital myasthenic syndrome due to DOK7 mutations. Neuromuscul Disord 19, 828-832 (2009).
19. A. Tucci, E. Kara, A. Schossig, N. I. Wolf, V. Plagnol, K. Fawcett, C. Paisan-Ruiz, M. Moore, D. Hernandez, S. Musumeci, M. Tennison, R. Hennekam, S. Palmeri, A. Malandrini, S. Raskin, D. Donnai, C. Hennig, A. Tzschach, R. Hordijk, T. Bast, K. Wimmer, C. N. Lo, S. Shorvon, H. Mefford, E. E. Eichler, R. Hall, I. Hayes, J. Hardy, A. Singleton, J. Zschocke, H. Houlden, Kohlschutter-Tonz syndrome: mutations in ROGDI and evidence of genetic heterogeneity. Hum Mutat 34, 296-300 (2013).
20. K. Bertram, D. E. Agafonov, O. Dybkov, D. Haselbach, M. N. Leelaram, C. L. Will, H. Urlaub, B. Kastner, R. Luhrmann, H. Stark, Cryo-EM Structure of a Pre-catalytic Human Spliceosome Primed for Activation. Cell 170, 701-713 e711 (2017).
21. F. Gualandi, E. Manzati, P. Sabatelli, C. Passarelli, M. Bovolenta, C. Pellegrini, D. Perrone, S. Squarzoni, E. Pegoraro, P. Bonaldo, A. Ferlini, Antisense-induced messenger depletion corrects a COL6A2 dominant mutation in Ullrich myopathy. Hum Gene Ther 23, 1313-1318 (2012).
22. M. Pertea, X. Lin, S. L. Salzberg, GeneSplicer: a new computational method for splice site prediction. Nucleic Acids Res 29, 1185-1190 (2001).

1.-8. (canceled)

9. A method for determining the likelihood that a genetic variant of a genetic locus defines a genetic disease or cancer-associated allele, wherein the genetic variant is located between a genomic sequence encoding a pre-mRNA 5′ splice-site and a genomic sequence encoding a related pre-mRNA branch-point site, the related branch-point site being operable with the 5′ splice-site in individuals who do not have the genetic disease or cancer to form an intron lariat in pre-mRNA transcribed from the locus, the method comprising:
determining whether a pre-mRNA transcribed from the locus would comprise a sufficient number of nucleotides between the 5′ splice-site and related branch-point site to enable formation of an intron lariat defined by the 5′ splice-site and related branch-point site;
wherein:
where the number of nucleotides between the 5′ splice-site and related branch-point site is insufficient for formation of an intron lariat defined by the 5′ splice-site and related branch-point site, a high likelihood that the genetic variant defines a genetic disease or cancer-associated allele is determined; and
where the number of nucleotides between the 5′ splice-site and related branch-point site is sufficient for formation of an intron lariat defined by the 5′ splice-site and related branch-point site, a low likelihood that the genetic variant defines a genetic disease or cancer-associated allele is determined;
thereby determining the likelihood that the genetic variant of the genetic locus defines a genetic disease or cancer-associated allele

10. The method of claim 9, wherein the genetic variant is a variant of uncertain significance (VUS).

11. The method of claim 9, wherein the 5′ splice-site or related branch-point site is not comprised in the genetic variant.

12. The method of claim 9, wherein the genetic variant results in an intragenic distance between the genomic sequence encoding the pre-mRNA 5′ splice-site and the genomic sequence encoding the related pre-mRNA branch-point site, that is from 1 to 5000 nucleotides shorter than the distance between the genomic sequence encoding the pre-mRNA 5′ splice-site and the genomic sequence encoding the respective pre-mRNA branch-point site in an individual who does not have the relevant genetic disease or cancer.

13. The method of claim 9, wherein the genetic variant results in an intragenic distance between the genomic sequence encoding the pre-mRNA 5′ splice-site and the genomic sequence encoding the related pre-mRNA branch-point site that is from, preferably 1 to 500 nucleotides shorter than the distance between the genomic sequence encoding the pre-mRNA 5′ splice-site and the genomic sequence encoding the respective pre-mRNA branch-point site in an individual who does not have the relevant genetic disease or cancer.

14. The method of claim 9, wherein the genetic variant results in an intragenic distance between the genomic sequence encoding the pre-mRNA 5′ splice-site and the genomic sequence encoding the related pre-mRNA branch-point site that is from 1 to 50 nucleotides shorter than the distance between the genomic sequence encoding the pre-mRNA 5′ splice-site and the genomic sequence encoding the respective pre-mRNA branch-point site in an individual who does not have the relevant genetic disease or cancer.

15. The method of claim 9, wherein the genetic variant comprises a deletion of a sequence of nucleotides.

16. The method of claim 9, wherein the genetic variant comprises a deletion of a sequence of nucleotides of from 1 to 50, 1 to 25, or 1 to 10 nucleotides.

17. The method of claim 9, wherein the genetic variant comprises a nucleotide insertion, a nucleotide sequence insertion and/or nucleotide substitution.

18. The method of claim 9, wherein the genomic sequence is assessed to determine whether a pre-mRNA transcribed from the locus would comprise a sufficient number of nucleotides between the 5′ splice-site and related branch-point site to enable formation of an intron lariat defined by the 5′ splice-site and related branch-point site.

19. The method of claim 9, wherein the genetic variant is located between genomic sequence encoding a pre-mRNA 5′ splice-site that may be cleaved by a U1/U2-dependent spliceosome complex and a genomic sequence encoding pre-mRNA related branch-point site that may be cleaved by a U1/U2-dependent spliceosome complex.

20. A method of treating an individual to minimise the likelihood of development or onset of a genetic disease or cancer,
wherein a genetic locus of the individual that controls or is associated with the disease comprises an allele comprising a genetic variant between a genomic sequence encoding a 5′ splice-site and a genomic sequence encoding a related branch-point site, the related branch-point site being operable with the 5′ splice-site in individuals who do not have genetic disease or cancer to form an intron lariat in pre-mRNA transcribed from the locus, the method comprising:
providing or having provided a test sample obtained from an individual for whom likelihood of development or onset of the genetic disease or cancer is to be minimised;
determining or having determined whether a pre-mRNA transcribed from the locus would comprise a sufficient number of nucleotides between the 5′ splice-site and related branch-point site to enable formation of an intron lariat defined by the 5′ splice-site and related branch-point site;
wherein:
where the number of nucleotides between the 5′ splice-site and related branch-point site is insufficient for formation of an intron lariat defined by the 5′ splice-site and related branch-point site, the method comprises administering a pharmaceutical compound to the individual for treatment of the genetic disease or cancer; and
where the number of nucleotides between the 5′ splice-site and related branch-point site is sufficient for formation of an intron lariat defined by the 5′ splice-site and related branch-point site, the method comprises not administering a pharmaceutical compound to the individual for treatment of the genetic disease or cancer;
thereby treating the individual for said genetic disease.

21. The method of claim 20, wherein the genetic variant is a variant of uncertain significance (VUS).

22. The method of claim 20, wherein the 5′ splice-site or related branch-point site is not comprised in the genetic variant.

23. The method of claim 20, wherein the genetic variant results in an intragenic distance between the genomic sequence encoding the pre-mRNA 5′ splice-site and the genomic sequence encoding the related pre-mRNA branch-point site that is from 1 to 5000, 1 to 500, or 1 to 50 nucleotides shorter than the distance between the genomic sequence encoding the pre-mRNA 5′ splice-site and the genomic sequence encoding the respective pre-mRNA branch-point site in an individual who does not have the relevant genetic disease or cancer.

24. The method of claim 20, wherein the genetic variant comprises a deletion of a sequence of nucleotides.

25. The method of claim 20, wherein the genetic variant comprises a deletion of a sequence of from 1 to 50, 1 to 25, or 1 to 10 nucleotides.

26. The method of claim 20, wherein the genetic variant comprises a nucleotide insertion, a nucleotide sequence insertion and/or nucleotide substitution.

27. The method of claim 20, wherein the genomic sequence is assessed to determine whether a pre-mRNA transcribed from the locus would comprise a sufficient number of nucleotides between the 5′ splice-site and related branch-point site to enable formation of an intron lariat defined by the 5′ splice-site and related branch-point site.

28. The method of claim 20, wherein the genetic variant is located between genomic sequence encoding a pre-mRNA 5′ splice-site that may be cleaved by a U1/U2-dependent spliceosome complex and a genomic sequence encoding pre-mRNA related branch-point site that may be cleaved by a U1/U2-dependent spliceosome complex.


SEQUENCE LISTING (11 sequences)

SEQ ID NO: 1 - DNA, 6 bp, Artificial Sequence
  Artificial nucleic acid sequence
acagag

SEQ ID NO: 2 - DNA, 19 bp, Artificial Sequence
  DOK7 primer 5′UTR F1
cgcggaaccatgacagaag

SEQ ID NO: 3 - DNA, 20 bp, Artificial Sequence
  DOK7 primer 5′ UTR F2
ttttgaaagtgaccctgggc

SEQ ID NO: 4 - DNA, 19 bp, Artificial Sequence
  DOK7 Primer exon-3R
tgggacaggcagacaatgg

SEQ ID NO: 5 - DNA, 20 bp, Artificial Sequence
  EMD primer exon-3F
cttcccaagaaagaggacgc

SEQ ID NO: 6 - DNA, 22 bp, Artificial Sequence
  EMD primer exon-6R1
gtgagccatgaagaggaagatg

SEQ ID NO: 7 - DNA, 18 bp, Artificial Sequence
  EMD primer exon-6R2
cctggcgatcctggccca

SEQ ID NO: 8 - DNA, 20 bp, Artificial Sequence
  EMD primer exon-5/6F
gagtgcaaggatagggaacg

SEQ ID NO: 9 - DNA, 20 bp, Artificial Sequence
  antisense DNA oligonucleotide
ccaaattcaccctgtgtagg

SEQ ID NO: 10 - DNA, 42 bp, Artificial Sequence
  COL6A-T7-F1 primer
acctaatacgactcactatagggtgcccatgatgctttgagg

SEQ ID NO: 11 - DNA, 21 bp, Artificial Sequence
  COL6A-R1 primer
atgcctctgtgagaccagtcc